(19) United States
(12) Patent Application Publication
(10) Pub. No.: US 2024/0052270 A1
(43) Pub. Date: Feb. 15, 2024

(54) LIQUID COMPOSITION COMPRISING PEPTIDE ALDEHYDE

(71) Applicant: BASF SE, Ludwigshafen am Rhein (DE)

(72) Inventors: Stephan HUEFFER, Ludwigshafen (DE); Sonja KUEBELBECK, Ludwigshafen (DE); Gabriele BOENEMANN, Ludwigshafen (DE); Katrin-Stephanie TUECKING, Ludwigshafen (DE); Grit BAIER, Ludwigshafen (DE); Stefan JENEWEIN, Ludwigshafen (DE); Stefan FISCHER, Lampertheim (DE)

(21) Appl. No.: 18/245,849

(22) PCT Filed: Sep. 17, 2021
(86) PCT No.: PCT/EP2021/075659

§ 371 Date: Mar. 17, 2023

(30) Foreign Application Priority Data
Sep. 22, 2020 (EP) 20197482.1

(51) Int. Cl. C11D 3/386; C11D 3/43; C11D 3/33; C11D 11/00; C11D 3/20

(52) U.S. Cl. CPC C11D 3/38663; C11D 3/43; C11D 3/33; C11D 11/0017; C11D 11/0023; C11D 3/2044; C11D 3/2093; C11D 3/38636

(57) ABSTRACT

A liquid composition, including
component (a): at least one peptide aldehyde, and
component (b): at least two organic solvents, where one organic solvent is 1,2-propane diol and at least one further organic solvent is selected from the group consisting of diols other than 1,2-propane diol in a weight ratio of 1,2-propane diol: at least one diol other than 1,2-propane diol of 25:1 to 1:4.

Enzyme stabilizers are usually employed to stabilize enzymes in liquid products. To inhibit enzyme activity temporarily, reversible enzyme inhibitors may be used, which are released in final application of an enzyme but are kept bound to the enzyme under storage conditions.

There is a request to provide liquid products which are homogenous at a temperature of about 20° C., and normal pressure of about 101.3 kPa. Homogenous means that the liquid composition does not show visible particles or turbidity. Specifically, a challenge is to provide water-containing products comprising enzyme stabilizers with limited solubility in water and/or water-miscible organic solvents.

The request extends to the storage stability of said homogenous products. Storage-stability/storage-stable in the context of homogeneity of the product means that the liquid composition does not show visible particles or turbidity after storage of the liquid composition at 4° C., 30° C. or 37° C.

Specifically, there is a need to provide liquid compositions comprising at least one hydrolase. An even more preferred request relates to providing homogenous liquid enzyme containing product, wherein the hydrolase comprised is storage stable. Storage-stability/storage-stable in the context of hydrolase stability means that at least one hydrolase comprised in the hydrolase containing product shows sufficient enzyme activity after storage at elevated temperatures, at 30° C. or 37° C. for 28 days or more. Sufficient enzyme activity usually means that after storage of a liquid enzyme containing product there is sufficient residual enzymatic activity when compared to the initial enzymatic activity that was available before storage.

In one aspect, the object was to find a solvent system, which allows dissolution of at least one enzyme stabilizer having limited solubility in water and/or water-miscible organic solvents in a water-containing hydrolase product. A further objective of the current invention was to provide a homogenous liquid composition comprising at least one enzyme stabilizer having limited solubility in water and/or water-miscible organic solvents which is suitable to be combined with at least one hydrolase.

The current invention provides liquid compositions, comprising
component (a): at least one enzyme stabilizer, preferred a peptide aldehyde, even more preferably a tripeptide aldehyde and
component (b): a mixture of at least two organic solvents, wherein preferably at least one of the organic solvents is 1,2-propane diol.

The composition of the invention is liquid at 20° C. and 101.3 kPa.

The liquid compositions of the invention are homogenous at ambient temperature and retain homogeneity during storage at 4° C., 30° C. or 37° C. for ≥50 days.

In one embodiment, the liquid compositions of the invention are provided in combination with at least one hydrolase (component (c)), preferably comprising a catalytic triad having the motif serine-histidine-aspartate as disclosed herein.

Preferably, the liquid compositions are storage-stable in terms of homogeneity and residual enzyme activity.

In one aspect, the components of the liquid composition are added as a composition or individually to at least one detergent component to provide liquid detergent formulations, wherein the liquid detergent formulation is preferably comprising at least one hydrolase (component (c)), preferably comprising a catalytic triad having the motif serine-histidine-aspartate as disclosed herein, and wherein said detergent formulation is storage-stable.

In one aspect the invention therefor relates to a detergent formulation comprising
component (a): at least one enzyme stabilizer, preferred a peptide aldehyde, even more preferably a tripeptide aldehyde and
component (b): a mixture of at least two organic solvents, wherein preferably at least one of the organic solvents is 1,2-propane diol and
component (c): at least one enzyme selected from the group of hydrolases (EC 3), preferably comprising a catalytic triad having the motif serine-histidine-aspartate as disclosed herein
and at least one detergent component.

In one embodiment, the detergent formulations are liquid.

The liquid detergent formulations of the invention are preferably storage-stable. Storage-stability in the context of liquid detergent formulations (which may be called in-detergent storage stability herein) comprising at least one hydrolase (component (c), preferably comprising a catalytic triad having the motif serine-histidine-aspartate as disclosed herein) relate to the storage-stability of the hydrolase at 30° C. or 37° C., meaning that sufficient residual enzymatic activity is available after storage when compared to the initial enzymatic activity that was available before storage.

### DETAILED DESCRIPTION

The current invention provides a homogenous liquid composition, comprising
component (a): at least one enzyme stabilizer, preferred a peptide aldehyde, even more preferably a tripeptide aldehyde
component (b): a mixture of at least two organic solvents, wherein preferably at least one of the organic solvents is 1,2-propane diol.

The composition of the invention is liquid at 20° C. and 101.3 kPa.

Component (a)

Component (a) comprises at least one enzyme stabilizer.

At least one enzyme stabilizer is selected from boron-containing compounds and peptide stabilizers.

Boron-containing compounds may be selected from boric acid or its derivatives and from boronic acid or its derivatives such as aryl boronic acids or its derivatives, from salts thereof, and from mixtures thereof. Boric acid herein may be called orthoboric acid. In one embodiment, boron-containing compound is selected from the group consisting of benzene boronic acid (BBA) which is also called phenyl boronic acid (PBA), derivatives thereof, and mixtures thereof. Preferably, phenyl-boronic acid derivatives are selected from the group consisting of 4-formyl phenyl boronic acid (4-FPBA), 4-carboxy phenyl boronic acid (4-CPBA), 4-(hydroxymethyl) phenyl boronic acid (4-HMPBA), and p-tolylboronic acid (p-TBA). Preferably, liquid compositions of the invention comprise 4-formyl phenyl boronic acid (4-FPBA).

In one embodiment, the liquid compositions of the invention are free from boron-containing compounds.

In one embodiment, component (a) comprises at least one peptide stabilizer, meaning peptide aldehyde herein. At least one peptide aldehyde is selected from di-, tri- or tetrapeptide aldehydes and aldehyde analogues (either of the form B1-BO—R wherein, R is H, CH₃, CX₃, CHX₂, or CH₂X (X=halogen), BO is a single amino acid residue (in one embodiment with an optionally substituted aliphatic or aromatic side chain); and B1 consists of one or more amino acid residues (in one embodiment one, two or three), optionally comprising an N-terminal protection group, or as described in WO 09/118375 and WO 98/13459, or a protease inhibitor of the protein type such as RASI, BASI, WASI (bifunctional alpha-amylase/subtilisin inhibitors of rice, barley and wheat) or Cl₂ or SSI.

In a preferred embodiment, the peptide aldehyde is a tripeptide aldehyde, preferably selected from a compound of formula (PA) or a salt thereof or hydrosulfit adduct thereof:

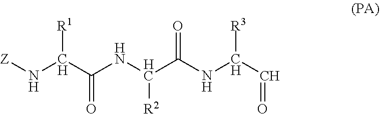

R¹, R², R³ and Z within formula (PA) are defined as follows:
R¹ is a group such that NH—CHR¹—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle. Preferably, R¹ is a group such that NH—CHR¹—CO is an L or D-amino acid residue of Ala, Val, Gly, Arg, Leu, Phe, Ile, His or Thr. More preferably, R¹ is a group such that NH—CHR¹—CO is an L or D-amino acid residue of Ala, Val, Gly, Arg, Leu, Ile or His.
R² is a group such that NH—CHR²—CO is an L or D-amino acid residue of Gly, Ala, Val, Leu, Ile, Met, Pro, Phe, Trp, Ser, Thr, Asp, Gln, Tyr, Cys, Lys, Arg, His, Asn, Glu, m-tyrosine, 3,4-dihydroxyphenylalanine, Nva, or Nle. Preferably, R² is a group such that NH—CHR²—CO is an L or D-amino acid residue of Ala, Cys, Gly, Pro, Ser, Thr, Val, Nva or Nle. More preferably, R² is a group such that NH—CHR²—CO is an L or D-amino acid residue of Ala, Gly, Pro or Val.
R³ is a group such that NH—CHR³—CO is an L or D-amino acid residue of Tyr, m-tyrosine, 3,4-dihydroxyphenylalanine, Phe, Val, Ala, Met, Nva, Leu, Ile or Nle or other non-natural amino acids carrying alkyl groups. Preferably, R³ is a group such that NH—CHR³—CO is an L or D-amino acid residue of Tyr, Phe, Val, Ala or Leu.

R¹ and R² may be groups such that NH—CHR¹—CO and/or NH—CHR²—CO are non-polar amino acid residues which herein include amino acids comprising aliphatic or aromatic R groups. Nonpolar amino acids herein specifically include Gly, Ala, Val, Leu, Ile, Met, Pro, Phe and Trp.

In one embodiment, R¹ and R² are groups such that NH—CHR¹—CO and/or NH—CHR²—CO are non-polar amino acids, preferably independently from each other selected from an L or D-amino acid residue of Ala, Val, Gly and Leu. R³ is a group such that NH—CHR³—CO is an L or D-amino acid residue of Tyr, Phe, Val, Ala or Leu.

In one embodiment, R¹ is a group such that NH—CHR¹—CO is an L or D-amino acid residue of Gly or Val, R² is a group such that NH—CHR²—CO is an L or D-amino acid residue of Ala, and R³ is a group such that NH—CHR³—CO is an L or D-amino acid residue of Tyr, Ala, or Leu.

In one embodiment, at least two selected from R¹, R² and R³ are groups such that NH—CHR¹—CO and/or NH—CHR²—CO and/or NH—CHR³—CO are non-polar amino acid residues, preferably independently from each other selected from an L or D-amino acid residue of Ala, Val, Gly and Leu.

In one embodiment, R¹ is a group such that NH—CHR¹—CO is an L or D-amino acid residue of Val, R² is a group such that NH—CHR²—CO is an L or D-amino acid residue of Ala, and R³ is a group such that NH—CHR³—CO is an L or D-amino acid residue of Leu.

The more non-polar amino acids are comprised in the peptide aldehyde, the more challenges exist to solubilize the peptide stabilizer in water and/or water-miscible solvents such as 1,2-propane diol and MPEG (methoxy polyethylene glycol).

Z in formula PA is selected from hydrogen, an N-terminal protection group, and one or more amino acid residues optionally comprising an N-terminal protection group. Preferably, Z is an N-terminal protection group.

If Z is one or more amino acid residue(s) comprising an N-terminal protection group, the N-terminal protection group is preferably a small aliphatic group, e.g., formyl, acetyl, fluorenylmethyloxycarbonyl (Fmoc), tert-butyloxycarbonyl (Boc), methoxycarbonyl (Moc); methoxyacetyl (Mac); methyl carbamate or a methylamino carbonyl/methyl urea group. In the case of a tripeptide, the N-terminal protection group is preferably a bulky aromatic group such as benzoyl (Bz), benzyloxycarbonyl (Cbz), p-methoxybenzyl carbonyl (MOZ), benzyl (Bn), p-methoxybenzyl (PMB) or p-methoxyphenyl (PMP).

Further suitable N-terminal protection groups are described in Greene's Protective Groups in Organic Synthesis, Fifth Edition by Peter G. M. Wuts, published in 2014 by John Wiley & Sons, Inc and in Isidro-Llobet et al., Amino Acid-Protecting Groups, Chem. Rev. 2009 109(6), 2455-2504.

In a preferred embodiment, the liquid compositions of the invention comprise at least one peptide aldehyde (component (a)) selected from compounds according to formula (PA), wherein
R¹ is a group such that NH—CHR¹—CO is an L or D-amino acid residue of Gly or Val, R² is a group such that NH—CHR²—CO is an L or D-amino acid residue of Ala, and R³ is a group such that NH—CHR³—CO is an L or D-amino acid residue of Tyr, Ala, or Leu. and
the N-terminal protection group Z is selected from benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), or tert-butyloxycarbonyl (Boc),

and wherein at least one peptide aldehyde is comprised in amounts in the range of about 0.05% to 0.8% by weight relative to the total weight of the liquid composition, wherein the amount relates to 100% active content. Preferably, the peptide aldehyde is comprised in amounts in the range of about 0.1% to 0.6% by weight, of about 0.12% to 0.5% by weight, of about 0.15% to 0.4%, or of about 0.2% to 0.35% by weight, all relative to the total weight of the liquid composition.

In a more preferred embodiment, at least one peptide aldehyde is selected from compounds according to formula (PA), wherein
R¹ is a group such that NH—CHR¹—CO is an L or D-amino acid residue of Val, R² is a group such that NH—CHR²—CO is an L or D-amino acid residue of Ala, and R³ is a group such that NH—CHR³—CO is an L or D-amino acid residue of Leu.

Even more preferably, the N-terminal protection group Z of the peptide aldehyde is benzyloxycarbonyl (Cbz).

Component (b)

Component (b) comprises at least two diols, preferably both with a boiling point of ≥130° C.

In one embodiment, component (b) comprises 1,2-propane diol (i.e. MPG, monopropylene glycol) and at least one “diol (other than 1,2-propane diol)” having a boiling point of 170° C. or ≥1190° C. More preferably, the boiling point of the second organic solvent is ≥205° C. The boiling points herein mean those at 101.3 kPa.

At least one “diol (other than 1,2-propane diol)” comprised in component (b) is preferably water-miscible.

“Water-miscibility/water-miscible” in this context means the property of the “diol (other than 1,2-propane diol)” to mix in those proportions in water relevant for this invention, forming a homogeneous solution. “Water-miscibility/water-miscible” preferably relates to the property at a temperature range of ambient temperatures to the melting point of the second organic solvent.

Preferably, “diol (other than 1,2-propane diol)” having a boiling point of 170° C. are selected from “alpha-omega diols”, 1,2 butane-diol, 1,3-butane diol, 1,2-pentane diol, and 1,2-hexane diol. In one embodiment, at least one “diol (other than 1,2-propane diol)” is selected from 1,2-pentane diol and 1,2-hexane diol. In another embodiment, at least one “diol (other than 1,2-propane diol)” is selected from “alpha-omega diols”.

“Alpha-omega diols” means compounds carrying two hydroxyl groups, each located at one of the ends of a linear molecule (HO—R—OH). Preferably, alpha-omega diols are liquid at temperatures of about 55° C. or 50° C. and at a pressure of 101.3 kPa.

Alpha-omega diols are preferably selected from compounds of formula: HO—R—OH, wherein R is C₃-C₇ alkylene or CH₂CH₂—O—CH₂CH₂ or CH₂CH(CH₃)—O—CH(CH₃)—CH₂, such as 1,3-propane diol, 1,4-butane diol, 1,6-hexane diol, diethylene glycol, and dipropylene glycol. More preferably, the alpha-omega diol is selected from 1,6-hexane diol and diethylene glycol. Most preferably, the alpha-omega diol is 1,6-hexane diol.

The liquid compositions of the invention preferably comprise total amounts of component (b) ranging from about 40% to 70% by weight, preferably from about 45% to 65% by weight. For example, the total amount of component (b) in the liquid compositions of the invention may be about 40% by weight, about 45% by weight, about 50% by weight, about 55% by weight, about 60% by weight, about 65% by weight, or about 70% by weight, all relative to the total weight of the liquid composition.

In one embodiment, liquid compositions comprise amounts of 1,2-propane diol in the range of about 10% to 60% by weight, of about 20% to 55% by weight, of about 20% to 60% by weight, of about 30% to 55% by weight, of about 20% to 30% by weight, all relative to the total weight of the liquid composition.

When the “diol (other than 1,2-propane diol)” is selected from 1,2-pentane diol and 1,2-hexane diol, the weight ratio of MPG:“diol (other than 1,2-propane diol)” is preferably in the range of 4:1 to 1:4, preferably at a total amount of component (b) in the liquid composition of 40% to 55% by weight relative to the total weight of the liquid composition.

Preferably, the liquid compositions comprise 1,2-pentane diol or 1,2-hexane diol in amounts in the range of about 10% to 40% by weight, at a total amount of component (b) in the liquid composition of 40% to 50% by weight. By weight means relative to the total weight of the liquid composition.

In one embodiment, the weight ratio of MPG:1,2-pentane diol is in the range of about 2.5:1 to 1:4. Preferably, the liquid composition comprises 1,2-pentane diol in amounts indicated before.

In one embodiment, the weight ratio of MPG:1,2-hexane diol is in the range of about 4:1 to 1:1.5. Preferably, the liquid composition comprises 1,2-hexane diol in amounts in the range of about 10% to 30% by weight relative to the total weight of the liquid composition.

When the “diol (other than 1,2-propane diol)” is selected from “alpha-omega diol”, the weight ratio of MPG:“diol (other than 1,2-propane diol)” is preferably in the range of 25:1 to 4:1, preferably at a total amount of component (b) in the liquid compositions of 50% to 60% by weight relative to the total weight of the liquid composition.

Preferably at least one “alpha-omega diol”, is comprised in liquid compositions of the invention in amounts in the range of about 1% to 10% by weight, preferably at a total amount of component (b) in the liquid compositions of 50% to 60% by weight, all relative to the total weight of the liquid composition.

“Alpha-omega diol” is preferably selected from 1,6-hexane diol and diethylene glycol.

In one embodiment, the weight ratio of MPG:1,6-hexane diol is in the range of about 25:1 to 4.5:1, more preferably 12:1 to 4.5:1. Preferably, the liquid composition comprises 1,6-hexane diol in amounts in the range of about 1% to 10% by weight, preferably at a total amount of component (b) in the liquid compositions of 50% to 60% by weight. By weight means relative to the total weight of the liquid composition.

In one embodiment, the weight ratio of MPG:diethylene glycol is in the range of about 10:1. Preferably, the liquid composition comprises diethylene glycol in amounts in the range of about 1% to 10% by weight, preferably of about 5% by weight. Preferably said amounts apply at a total amount of component (b) in the liquid composition of about 55% by weight. By weight means relative to the total weight of the liquid composition.

In one embodiment, the weight ratio of “diol (other than 1,2-propane diol)”:component (a) is in the range of about 100:1 to about 50:1, when “diol (other than 1,2-propane diol)” is selected from 1,2-pentane diol and 1,2-hexane diol, preferably at further ratios, amounts, combinations and total amounts as indicated in the paragraphs before.

In one embodiment, the weight ratio of “diol (other than 1,2-propane diol)”:component (a) is in the range of about 40:1 to about 10:1, when “diol (other than 1,2-propane diol)” is selected from “alpha-omega diol”, preferably selected from 1,6-hexane diol and diethylene glycol, preferably at further ratios, amounts, combinations and total amounts as indicated in the paragraphs before.

In one embodiment, the weight ratio of 1,6-hexane diol:component (a) in the liquid composition of the invention is ranging from about 30:1 to about 10:1, preferably 25:1 to 20:1, preferably at further ratios, amounts, combinations and total amounts as indicated in the paragraphs before.

In one preferred embodiment, the liquid compositions comprise 1,2-propane diol and 1,2-pentane diol in a weight ratio of about 12:1 to 7:1; preferably, 1,2-propane diol is comprised in amounts of about 30% to 35% by weight relative to the total weight of the liquid composition; preferably at further ratios, amounts, combinations and total amounts as indicated in the paragraphs before.

In one embodiment, the liquid compositions of the invention are free from surfactants. “Free from surfactants” means, that less than about 10% by weight, less than about 8%, less than about 6%, less than about 4%, less than about 2% or essentially no surfactants are comprised in the liquid composition relative to the total weight of the liquid composition. “Surfactants” in the context of the liquid compositions of the invention mean total surfactant comprised.

The liquid compositions of the invention are preferably free from non-phosphate based builders. “Free from non-phosphate based builders” means, that less than about 3%, preferably less than about 2% and more preferably less than about 1% by weight or essentially no non-phosphate based builders are comprised in the liquid composition relative to the total weight of the liquid composition. “Non-phosphate based builders” in the context of the liquid compositions of the invention means total amount of non-phosphate based builders comprised. The non-phosphate based builders are preferably selected from aminocarboxylates, citrate and phosphonates as disclosed herein. “Essentially no non-phosphate based builders means” amounts of nonphosphate builder is below 0.1% by weight, preferably 0% by weight, all relative to the total weight of the liquid composition.

In one embodiment, the liquid compositions of the invention are free from surfactants and free from non-phosphate based builders.

The liquid compositions, in one embodiment, comprise a at least one preservative. Preferably, preservative means substances that are added to a liquid composition for the purpose of preservation, meaning more preferably that compounds known to have preserving features comprised in a liquid composition formed in the production process are excluded from the term preservatives. In one embodiment, at least one preservative is selected from the group consisting of 2-phenoxyethanol, glutaraldehyde, 2-bromo-2-nitropropane-1,3-diol, and formic acid in acid form or as its salt, and 4,4′-dichloro 2-hydroxydiphenylether. Usually, the liquid compositions of the invention comprise at least one preservative in amounts below 10 ppm, such as in amounts ranging from 2 ppm to 5% by weight relative to the total weight of the liquid composition. In one embodiment, the liquid compositions are free from preservatives, meaning that preservatives are comprised in amounts less than 1 ppm.

Component (c)

In a preferred embodiment, the liquid compositions further comprise at least one hydrolase (component (c)). Component (c) comprises at least one hydrolase (EC 3). Preferred enzymes are selected from the group of enzymes acting on ester bond (E.C. 3.1), glycosylases (E.C. 3.2), and peptidases (E.C. 3.4). Enzymes acting on ester bond (E.C. 3.1), are hereinafter also referred to as lipases, and DNAses. Glycosylases (E.C. 3.2) are hereinafter also referred to as either amylases, cellulases, or mannanases. Peptidases (E.C. 3.4) are hereinafter also referred to as proteases.

Hydrolases comprised in component (c) are identified by polypeptide sequences (also called amino acid sequences herein). The polypeptide sequence specifies the three-dimensional structure including the “active site” of an enzyme which in turn determines the catalytic activity of the same. Polypeptide sequences may be identified by a SEQ ID NO. According to the World Intellectual Property Office (WIPO) Standard ST.25 (1998) the amino acids herein are represented using three-letter code with the first letter as a capital or the corresponding one letter.

Any enzyme comprised in component (c) according to the invention relates to
a parent polypeptide (sequence) or amino acid sequence and/or
a variant parent polypeptide (sequence) or amino acid sequence,

both having enzymatic activity. Proteins or polypeptides having enzymatic activity are enzymatically active or exert enzymatic conversion, meaning that enzymes act on substrates and convert these into products. The term “enzyme” herein excludes inactive variants of an enzyme.

A “parent” sequence (of a parent protein or polypeptide, also called “parent enzyme”) is the starting sequence for introduction of changes (e.g. by introducing one or more amino acid substitutions, insertions, deletions, or a combination thereof) to the sequence, resulting in “variants” of the parent sequences. The term parent enzyme (or parent sequence) includes wild-type enzymes (sequences) and synthetically generated sequences (enzymes) which are used as starting sequences for introduction of (further) changes.

The term “enzyme variant” or “sequence variant” or “variant enzyme” refers to an enzyme that differs from its parent enzyme in its amino acid sequence to a certain extent. If not indicated otherwise, variant enzyme “having enzymatic activity” means that this variant enzyme has the same type of enzymatic activity as the respective parent enzyme.

In describing enzyme variants usually substitutions, deletions and insertions occur when compared to a parent sequence. Herein nomenclature is used known to those skilled in the art. Amino acid substitutions are usually described by providing the original amino acid followed by the number of the position within the amino acid sequence, followed by the substituted amino acid. Amino acid deletions are usually described by providing the original amino acid followed by the number of the position within the amino acid sequence, followed by *. Amino acid insertions are usually described by providing the original amino acid followed by the number of the position within the amino acid sequence, followed by the original amino acid and the additional amino acid. Where different alterations can be introduced at a position, the different alterations are separated by a comma.

Enzyme variants are usually defined by their sequence identity when compared to a parent enzyme. Sequence identity usually is provided as “% sequence identity” or “% identity”. For calculation of sequence identities, in a first step a sequence alignment has to be produced. According to this invention, a pairwise global alignment has to be produced, meaning that two sequences have to be aligned over their complete length, which is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (gap open=10.0, gap extend=0.5 and matrix=EBLOSUM62).

According to this invention, the following calculation of %-identity applies: %-identity=(identical residues/length of the alignment region which is showing the respective sequence of this invention over its complete length)*100.

According to this invention, enzyme variants are described as an amino acid sequence which is at least n % identical to the amino acid sequence of the respective parent enzyme with “n” being an integer between 10 and 100. In one embodiment, variant enzymes are at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical when compared to the full-length amino acid sequence of the parent enzyme, wherein the enzyme variant has enzymatic activity.

“Enzymatic activity” means the catalytic effect exerted by an enzyme, which usually is expressed as units per milligram of enzyme (specific activity) which relates to molecules of substrate transformed per minute per molecule of enzyme (molecular activity).

Variant enzymes have enzymatic activity according to the present invention when said enzyme variants exhibit at least 90%, at least 95%, or at least 100% of the enzymatic activity of the respective parent enzyme. Preferably, enzyme variants exhibit at least 100% of the enzymatic activity of the respective parent enzyme.

In one aspect of the invention, at least one enzyme comprised in component (c) is part of a liquid enzyme concentrate. “Liquid” in the context of enzyme concentrate is related to the physical appearance at 20° C. and 101.3 kPa. The liquid enzyme concentrate usually results from fermentation. Fermentation means the process of cultivating recombinant cells which express the desired enzyme in a suitable water-based nutrient medium allowing the recombinant host cells to grow and express the desired protein. At the end of the fermentation, fermentation broth is usually collected and liquid fraction is separated from solid fraction. Depending on whether the enzyme has been secreted into the liquid fraction or not, the desired protein or enzyme can be recovered from the liquid fraction of the fermentation broth or from cell lysates. Recovery of the desired enzyme uses methods known to those skilled in the art. Suitable methods for recovery of proteins or enzymes from fermentation broth include but are not limited to collection, centrifugation, filtration, extraction, and precipitation.

Liquid enzyme concentrates usually comprise amounts of enzyme up to 40% by weight, or up to 30% by weight, or up to 25% by weight, all relative to the total weight of the enzyme concentrate.

Enzyme concentrates which result from fermentation comprise water and potentially further residual components such as salts originating from the fermentation medium, cell debris originating from the production host cells, metabolites produced by the production host cells during fermentation. In one embodiment, residual components are comprised in liquid enzyme concentrates in amounts less than 20% by weight less, than 10% by weight, or less than 5% by weight, all relative to the total weight of the enzyme concentrate.

In one embodiment, component (c) comprises at least one hydrolase comprising as a catalytic triad the amino acids aspartate, histidine and serine. In one embodiment, component (c) comprises at least one hydrolase comprising a catalytic triad having a motif selected from aspartate-histidine-serine and serine-histidine-aspartate, wherein the relative order of these amino acids reads from the amino to carboxy-terminus.

In a preferred embodiment, the hydrolase (component (c)) comprises a catalytic triad having the motif serine-histidine-aspartate, wherein the relative order of these amino acids reads from the amino to carboxy-terminus. Preferably, said hydrolase (component (c)) is selected from proteases and lipases.

Protease

In one embodiment, component (c) comprises at least one protease.

In one embodiment, at least one protease is selected from the group consisting of serine endopeptidases (EC 3.4.21), most preferably chymotrypsin related proteases (3.4.21.1) and subtilisin type proteases (EC 3.4.21.62).

In one embodiment, component (c) comprises at least one protease comprising a catalytic triad having a motif selected from aspartate-histidine-serine and serine-histidine-aspartate, wherein the relative order of these amino acids reads from the amino to carboxy-terminus.

Subtilisins and chymotrypsin related serine proteases both have a catalytic triad comprising aspartate, histidine and serine. In the subtilisin related proteases the relative order of these amino acids, reading from the amino to carboxy-terminus is aspartate-histidine-serine. In the chymotrypsin related proteases the relative order, however is histidine-aspartate-serine.

Preferably, at least one protease comprises a catalytic triad comprising the amino acids aspartate-histidine-serine, wherein the relative order of these amino acids reads from the amino to carboxy-terminus. More preferably at least one protease is a subtilisin related protease.

In one embodiment of the present invention, component (c) comprises at least one protease selected from the following: subtilisin from Bacillus amyloliquefaciens BPN′ (described by Vasantha et al. (1984) J. Bacteriol. Volume 159, p. 811-819 and JA Wells et al. (1983) in Nucleic Acids Research, Volume 11, p. 7911-7925); subtilisin from Bacillus licheniformis (subtilisin Carlsberg; disclosed in E L Smith et al. (1968) in J. Biol Chem, Volume 243, pp. 2184-2191, and Jacobs et al. (1985) in Nucl. Acids Res, Vol 13, p. 8913-8926); subtilisin PB92 (original sequence of the alkaline protease PB92 is described in EP 283075 A2); subtilisin 147 and/or 309 (Esperase®, Savinase®, respectively) as disclosed in WO 89/06279; subtilisin from Bacillus lentus as disclosed in WO 91/02792, such as from Bacillus lentus DSM 5483 or the variants of Bacillus lentus DSM 5483 as described in WO 95/23221; subtilisin from Bacillus alcalophilus (DSM 11233) disclosed in DE 10064983; subtilisin from Bacillus gibsonii (DSM 14391) as disclosed in WO 2003/054184; subtilisin from Bacillus sp. (DSM 14390) disclosed in WO 2003/056017; subtilisin from Bacillus sp. (DSM 14392) disclosed in WO 2003/055974; subtilisin from Bacillus gibsonii (DSM 14393) disclosed in WO 2003/054184; subtilisin having SEQ ID NO: 4 as described in WO 2005/063974; subtilisin having SEQ ID NO: 4 as described in WO 2005/103244; subtilisin having SEQ ID NO: 7 as described in WO 2005/103244; and subtilisin having SEQ ID NO: 2 as described in application DE 102005028295.4.

In one embodiment, component (c) comprises at least subtilisin 309 (which might be called Savinase herein) as disclosed as sequence a) in Table I of WO 89/06279 or a variant thereof which is at least 80% similar and/or identical thereto and has proteolytic activity.

Examples of useful proteases in accordance with the present invention comprise the variants of subtilisin protease derived from SEQ ID NO:22 as described in EP 1921147 (which is the sequence of mature alkaline protease from Bacillus lentus DSM 5483) with amino acid substitutions in one or more of the following positions: 3, 4, 9, 15, 24, 27, 33, 36, 57, 68, 76, 77, 87, 95, 96, 97, 98, 99, 100, 101, 102, 103, 104, 106, 118,120, 123, 128, 129, 130,131, 154, 160, 167, 170, 194, 195, 199, 205, 206, 217, 218, 222, 224, 232, 235, 236, 245, 248, 252 and 274 (according to the BPN′ numbering), which have proteolytic activity. In one embodiment, such a protease is not mutated at positions Asp32, His64 and Ser221 (according to BPN′ numbering).

Component (c) preferably comprises at least one protease variant having proteolytic activity which is at least 85%, at least 90%, at least 91%, at least 92%, at least 93%, at least 94%, at least 95%, at least 96%, at least 97%, at least 98% or at least 99% identical when compared to the full-length polypeptide sequence of the parent enzyme as disclosed above.

In one embodiment, at least one protease comprised in component (c) has SEQ ID NO:22 as described in EP 1921147, or a protease which is at least 80% identical thereto and has proteolytic activity. In one embodiment, said protease is characterized by having at least amino acid glutamic acid (E), or aspartic acid (D), or asparagine (N), or glutamine (Q), or alanine (A), or glycine (G), or serine (S) at position 101 (according to BPN′ numbering) and has proteolytic activity. In one embodiment, said protease comprises one or more further substitutions: (a) threonine at position 3 (3T), (b) isoleucine at position 4 (4I), (c) alanine, threonine or arginine at position 63 (63A, 63T, or 63R), (d) aspartic acid or glutamic acid at position 156 (156D or 156E), (e) proline at position 194 (194P), (f) methionine at position 199 (199M), (g) isoleucine at position 205 (205I), (h) aspartic acid, glutamic acid or glycine at position 217 (217D, 217E or 217G), (i) combinations of two or more amino acids according to (a) to (h).

In one embodiment, at least one protease comprised in component (c) is at least 80% identical to SEQ ID NO:22 as described in EP 1921147 and is characterized by comprising one amino acid (according to (a)-(h)) or combinations according to (i) together with the amino acid 101E, 101D, 101N, 101Q, 101A, 101G, or 101S (according to BPN′ numbering) and having proteolytic activity. In one embodiment, said protease is characterized by comprising the mutation (according to BPN′ numbering) R101E, or S3T+V4I+V205I, or R101E and S3T, V4I, and V205I, or S3T+V4I+V199M+V205I+L217D, and having proteolytic activity.

In one embodiment, component (c) comprises at least one protease which is at least 80% identical to SEQ ID NO:22 as described in EP 1921147 and comprises the amino acid substitutions R101E and S156D and/or L262E, and optionally at least one further mutation selected from I104T, H120D, Q137H, S141H, R145H and S163G (according to BPN′ numbering).

In one embodiment, at least one protease comprised in component (c) is at least 80% identical to SEQ ID NO:22 as described in EP 1921147 and has at least the mutations
S3T+V4I+R101E+V205I or
S9R+A15T+V68A+N218D+Q245R and optionally at least one further mutation selected from S3T, V4I, D99S, R101S, A103S and I104V or
R101E+S156D+L262E and optionally at least one further mutation selected from I104T, H120D, Q137H, S141H, R145H and S163G,
all numberings according to the BPN′ numbering.

In one embodiment, protease according to SEQ ID NO:22 as described in EP 1921147 is characterized by comprising the mutations (according to BPN′ numbering) S3T+V4I+S9R+A15T+V68A+D99S+R101S+A103S+I104V+N218D and having proteolytic activity. In one embodiment, at least one protease comprised in component (c) is a protease 80% identical to SEQ ID NO:22 as described in EP 1921147 having R101E (according to BPN′ numbering). Preferably, at least one protease comprised in component (c) is a protease 100% identical to SEQ ID NO:22 as described in EP 1921147 having R101E (according to BPN′ numbering).

According to the present invention, component (c), in one embodiment, comprises a combination of at least two proteases, preferably selected from the group of serine endopeptidases (EC 3.4.21), more preferably selected from the group of subtilisin type proteases (EC 3.4.21.62)—all as disclosed above.

Proteases herein are active proteins exerting “protease activity” or “proteolytic activity”. Proteolytic activity is related to the rate of degradation of protein by a protease or proteolytic enzyme in a defined course of time. The methods for analyzing proteolytic activity are well-known in the literature (see e.g. Gupta et al. (2002), Appl. Microbiol. Biotechnol. 60: 381-395). Herein, proteolytic activity is determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage, resulting in release of yellow color of free pNA which can be quantified by measuring OD₄₀₅.

Proteolytic activity usually is provided in units per gram enzyme. For example, 1 U protease regularly is defined as the amount of protease which sets free 1 μmol folin-positive amino acids and peptides (as tyrosine) per minute at pH 8.0 and 37° C. (casein as substrate).

Lipase

In one embodiment, component (c) comprises at least one lipase.

“Lipases”, “lipolytic enzyme”, “lipid esterase”, all refer to an enzyme of EC class 3.1.1 (“carboxylic ester hydrolase”). Lipase means active protein having lipase activity (or lipolytic activity; triacylglycerol lipase, EC 3.1.1.3), cutinase activity (EC 3.1.1.74; enzymes having cutinase activity may be called cutinase herein), sterol esterase activity (EC 3.1.1.13) and/or wax-ester hydrolase activity (EC 3.1.1.50).

Preferably, at least one lipase comprises a catalytic triad comprises the amino acids aspartate-histidine-serine, wherein the relative order of these amino acids reads from the amino to carboxy-terminus is.

The methods for determining lipolytic activity are well-known in the literature (see e.g. Gupta et al. (2003), Biotechnol. Appl. Biochem. 37, p. 63-71). E.g. the lipase activity (lipolytic activity) may be measured by ester bond hydrolysis in the substrate para-nitrophenyl palmitate (pNP-Palmitate, C:16) and releases pNP which is yellow and can be detected at 405 nm.

In one embodiment, component (c) comprises at least one lipase selected from fungal triacylglycerol lipase (EC class 3.1.1.3). Fungal triacylglycerol lipase is selected from Thermomyces lanuginosa lipase. In one embodiment, Thermomyces lanuginosa lipase is selected from polypetides which are at least 90%, at least 91%, at least 92%, at least 93%, at least 94%, at least 95%, at least 96%, at least 97%, at least 98% or at least 99% identical when compared to the full-length polypeptide sequence of amino acids 1-269 of SEQ ID NO:2 of U.S. Pat. No. 5,869,438.

Thermomyces lanuginosa lipase is at least 80% identical to SEQ ID NO:2 of U.S. Pat. No. 5,869,438 characterized by having amino acid T231R and N233R. Said Thermomyces lanuginosa lipase preferably further comprise one or more of the following amino acid exchanges: Q4V, V60S, A150G, L227G, P256K.

Component (c), in one aspect, comprises at least one protease as disclosed above and/or at least one lipase as disclosed above.

In one embodiment the liquid composition comprises component (a) and component (c) in a weight ratio of about 15:1 to about 35:1, for example about 16:1, 20:1, 28:1, 30:1, 34:1.

In one embodiment, the liquid composition comprises component (c) in amounts in the range of about 3% to 8% by weight, of about 4% to 7%, or of about 4.5% to 6%.

In one embodiment, the molar ratio of component (a) to component (c) in liquid compositions of the invention ranges from 1:1 to 20:1. Preferably, component (a) to component (c) are present in the liquid compositions of the invention at a molar ratio of about 1:1 to about 10:1. In one embodiment, the molar ratio is 1:1 to 7:1. For example, the molar ratio may be about 7:1, about 5:1, about 4:1, about 3:1, about 2:1, or about 1:1.

Component (c) is comprised in liquid compositions which comprise component (a) and component (b) and their ratios, amounts and total amounts as defined above in the chapters on component (a) and component (b).

Component (d)

The liquid compositions of the invention, in one embodiment, comprise at least one water-soluble salt (component (d)). At least one water-soluble salt is preferably comprised in liquid compositions in amounts ranging from 0.1% to 0.5% by weight relative to the total weight of the liquid composition.

In one embodiment, component (d) comprises at least one compound selected from NaCl, KCl, alkali salts of lactic acid or formic acid, and mixtures thereof.

In one embodiment, the liquid compositions of the invention comprise water-soluble sources of zinc (II), calcium (II) and/or magnesium (II) ions in the finished compositions that provide such ions to the enzymes, as well as other metal ions (e.g. barium (II), scandium (II), iron (II), manganese (II), aluminum (III), Tin (II), cobalt (II), copper (II), Nickel (II), and oxovanadium (IV)).

In one embodiment, component (d) comprises CaCl₂, preferably in its hydrated form.

The liquid composition of the invention preferably comprises about 0.2% to 0.4% by weight, more preferably 0.25% to 0.35% by weight component (d), all relative to the total weight of the liquid composition.

Component (d) is comprised in liquid compositions which further comprise component (a), component (b) and component (c) and their ratios, amounts and total amounts as defined above in the chapters on component (a), component (b) and component (c).

In one embodiment, liquid compositions of the invention comprise
component (a): 0.05% to 0.8% by weight of at least one tripeptide aldehyde and
component (b): 40% to 70% by weight of a mixture of 1,2-propane diol and at least one “alpha-omega diol” in a weight ratio of 25:1 to 4:1 and
component (c): at least one protease (preferably subtilisin-type protease (EC 3.4.21.62)) and/or at least one lipase (preferably selected from selected from Thermomyces lanuginosa lipase) and
component (d): 0.1% to 0.5% by weight at least one water-soluble salt (preferably CaCl₂) and water up to 100% by weight
wherein the molar ratio of component (a) to component (c) ranges from 1:1 to 20:1 and
wherein % by weight is relative to the total weight of the liquid composition.

Preferably, said liquid compositions are free from surfactants and free from non-phosphate based builders.

Further Components

In one embodiment, the liquid compositions of the invention comprise besides a hydrolase (component (c)) comprising a catalytic triad having the motif serine-histidine-aspartate as disclosed herein, at least one further hydrolase selected from amylases, cellulases, mannanases, and DNAses. The liquid compositions may also comprise at least one other useful enzyme selected from pectate lyases and dispersins.

Amylase

In one embodiment, the liquid compositions of the invention comprise at least one amylase. “Amylases” according to the invention (alpha and/or beta) include those of bacterial or fungal origin (EC 3.2.1.1 and 3.2.1.2, respectively). Preferably, the liquid compositions of the invention comprise at least one alpha-amylase (EC 3.2.1.1). Chemically modified or protein engineered mutants are included.

Amylases comprised in the liquid compositions of the invention according to the invention have “amylolytic activity” or “amylase activity” involving (endo)hydrolysis of glucosidic linkages in polysaccharides. alpha-amylase activity may be determined by assays for measurement of alphaamylase activity which are known to those skilled in the art. Examples for assays measuring alpha-amylase activity are:
alpha-amylase activity can be determined by a method employing the Ethyliden-4-nitrophenyl-alpha-D-maltoheptaosid (EPS). D-maltoheptaoside is a blocked oligosaccharide which can be cleaved by an endo-amylase. Following the cleavage, the alpha-glucosidase included in the kit to digest the substrate to liberate a free PNP molecule which has a yellow color and thus can be measured by visible spectophotometry at 405 nm. The slope of the time dependent absorption-curve is directly proportional to the specific activity (activity per mg enzyme) of the alphaamylase in question under the given set of conditions.

At least one amylase comprised in the liquid compositions of the invention is preferably selected from the following:
amylases from Bacillus licheniformis having SEQ ID NO:2 as described in WO 95/10603. Suitable variants are described in WO 95/10603 comprising one or more substitutions in the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 which have amylolytic activity. Variants are described in WO 94/02597, WO 94/018314, WO 97/043424 and SEQ ID NO:4 of WO 99/019467.
amylases from B. stearothermophilus having SEQ ID NO:6 as disclosed in WO 02/10355 or an amylase with optionally having a C-terminal truncation over the wildtype sequence. Suitable variants of SEQ ID NO:6 include those comprising a deletion in positions 181 and/or 182 and/or a substitution in position 193.
amylases from Bacillus sp.707 having SEQ ID NO:6 as disclosed in WO 99/19467. Preferred variants of SEQ NO: 6 are those having a substitution, a deletion or an insertion in one or more of the following positions: R181, G182, H183, G184, N195, 1206, E212, E216 and K269.
amylases from Bacillus halmapalus having SEQ ID NO:2 or SEQ ID NO:7 as described in WO 96/23872, also described herein as SP-722. Preferred variants are described in WO 97/3296, WO 99/194671 and WO 2013/001078.
amylases from Bacillus sp. DSM 12649 having SEQ ID NO:4 as disclosed in WO 00/22103.
amylases from Bacillus strain TS-23 having SEQ ID NO:2 as disclosed in WO 2009/061380.
amylases from Cytophaga sp. having SEQ ID NO:1 as disclosed in WO 2013/184577.
amylases from Bacillus megaterium DSM 90 having SEQ ID NO:1 as disclosed in WO 2010/104675.
amylases from Bacillus sp. comprising amino acids 1 to 485 of SEQ ID NO:2 as described in WO 00/60060.
amylases from Bacillus amyloliquefaciens or variants thereof, preferably selected from amylases according to SEQ ID NO: 3 as described in WO 2016/092009.
amylases having SEQ ID NO:12 as described in WO 2006/002643 or amylase variants comprising the substitutions Y295F and M202LITV within said SEQ ID NO:12.
amylases having SEQ ID NO:6 as described in WO 2011/098531 or amylase variants comprising a substitution at one or more positions selected from the group consisting of 193 [G,A,S,T or M], 195 [F,W,Y,L,I or V], 197 [F,W,Y,L,I or V], 198 [Q or N], 200 [F,W,Y,L,I or V], 203 [F,W,Y,L,I or V], 206 [F,W,Y,N,L,I,V,H,Q,D or E], 210 [F,W,Y,L,I or V], 212 [F,W,Y,L,I or V], 213 [G,A,S,T or M] and 243 [F,W,Y,L,I or V] within said SEQ ID NO:6.
amylases having SEQ ID NO:1 as described in WO 2013/001078 or amylase variants comprising an alteration at two or more (several) positions corresponding to positions G304, W140, W189, D134, E260, F262, W284, W347, W439, W469, G476, and G477 within said SEQ ID NO:1.
amylases having SEQ ID NO:2 as described in WO 2013/001087 or amylase variants comprising a deletion of positions 181+182, or 182+183, or 183+184, within said SEQ ID NO:2, optionally comprising one or two or more modifications in any of positions corresponding to W140, W159, W167, Q169, W189, E194, N260, F262, W284, F289, G304, G305, R320, W347, W439, W469, G476 and G477 within said SEQ ID NO:2.
amylases which are hybrid alpha-amylases from above mentioned amylases as for example as described in WO 2006/066594;
hybrid amylases according to WO 2014/183920 with A and B domains having at least 90% similarity and/or identity to SEQ ID NO:2 of WO 2014/183920 and a C domain having at least 90% similarity and/or identity to SEQ ID NO:6 of WO 2014/183920, wherein the hybrid amylase has amylolytic activity; preferably the hybrid alpha-amylase is at least 95% similar and/or identical to SEQ ID NO: 23 of WO 2014/183920 and having amylolytic activity;
hybrid amylase according to WO 2014/183921 with A and B domains having at least 75% similarity and/or identity to SEQ ID NO: 2, SEQ ID NO: 15, SEQ ID NO: 20, SEQ ID NO: 23, SEQ ID NO: 29, SEQ ID NO: 26, SEQ ID NO: 32, and SEQ ID NO: 39 as disclosed in WO 2014/183921 and a C domain having at least 90% similarity and/or identity to SEQ ID NO: 6 of WO 2014/183921, wherein the hybrid amylase has amylolytic activity; preferably, the hybrid alpha-amylase is at least 95% similar and/or identical to SEQ ID NO: 30 as disclosed in WO 2014/183921 and having amylolytic activity.
hybrid amylase according to WO 2021/032881 comprising an A and B domain originating from the alpha amylase from Bacillus sp. A 7-7 (DSM 12368) and a C domain originating from the alpha amylase from Bacillus cereus wherein the hybrid amylase has amylolytic activity; preferably, the A and B domain are represented by SEQ ID NO: 42 and a C domain is represented by SEQ ID NO: 44—both sequences as disclosed in WO 2021/032881; more preferably, the hybrid amylase has a sequence according to SEQ ID NO: 54 as disclosed in WO 2021/032881.

According to the present invention, the liquid compositions of the invention, in one embodiment, comprise a combination of at least two amylases as disclosed above.

Cellulase

In one embodiment, the liquid compositions of the invention comprise at least one cellulase. “Cellulases”, “cellulase enzymes” or “cellulolytic enzymes” are enzymes involved in hydrolysis of cellulose. At least one cellulase is selected from cellobiohydrolase (1,4-β-glucan cellobiohydrolase, EC 3.2.1.91), endo-1,4-β-glucanase (endo-1,4-β-D-glucan 4-glucanohydrolase, EC 3.2.1.4) and β-glucosidase (EC 3.2.1.21). Preferably, the liquid compositions comprise at least one cellulase of the glycosyl hydrolase family 7 (GH7, pfam00840), preferably selected from endoglucanases (EC 3.2.1.4).

Assays for measurement of “cellulase activity” or “cellulolytic activity” are known to those skilled in the art. For example, cellulolytic activity may be determined by virtue of the fact that cellulase hydrolyses carboxymethyl cellulose to reducing carbohydrates, the reducing ability of which is determined colorimetrically by means of the ferricyanide reaction, according to Hoffman, W. S., J. Biol. Chem. 120, 51 (1937).

In one embodiment, the liquid compositions of the invention comprise at least one cellulase selected of the glycosyl hydrolase family 7 (GH7, pfam00840) lacking a CBD (carbohydrate binding domain), preferably selected from endoglucanases (EC 3.2.1.4) lacking a CBD (carbohydrate binding domain).

In one embodiment, at least one cellulase is selected from cellulases comprising a cellulose binding domain. In one embodiment, at least one cellulase is selected from cellulases comprising a catalytic domain only, meaning that the cellulase lacks cellulose binding domain.

In one embodiment, the liquid compositions of the invention comprise at least one cellulase originating from Humicola insolens DSM 1800, Bacillus sp, Thielavia terrestris, Fusarium oxysporum, Sordaria fimicola and Trichoderma reesei.

In one embodiment, the liquid compositions of the invention comprise at least one Humicola insolens DSM 1800 endoglucanase (EC 3.2.1.4) having the amino acid sequence as disclosed in position 21-435 of SEQ ID NO:2 of WO 2018/224544, and variants which are preferably at least 95% identical thereto.

In one embodiment, the liquid compositions of the invention comprise at least a Bacillus sp. cellulase (EC 3.2.1.4) selected from a polypeptide which is at least 80% similar and/or identical to the amino acid sequence of position 1 to position 773 of SEQ ID NO: 2 of WO 2004/053039 or a catalytically active fragment thereof. In one embodiment, at least one cellulase is a mature polypeptide which is at least 95% identical to an amino acid sequence according to SEQ ID NO:1 of WO 2018/224544.

In one embodiment, the liquid compositions of the invention comprise at least a Thielavia terrestris cellulase (EC 3.2.1.4) having a polypeptide at least 80% similar and/or identical to the amino acid sequence of position 1 to position 299 of SEQ ID NO: 4 of WO 2004/053039 or a catalytically active fragment thereof. In one embodiment, at least one cellulase is a mature polypeptide which is at least 95% identical to an amino acid sequence according to SEQ ID NO:4 of WO 2018/224544.

In one embodiment, at least one cellulase is a mature Sordaria fimicola cellulase which is at least 95% identical to an amino acid sequence according to SEQ ID NO:5 of WO 2018/224544.

According to the present invention, the liquid compositions of the invention, in one embodiment, comprise a combination of at least two cellulases, preferably selected from endoglucanases (EC 3.2.1.4) as disclosed above.

Mannanase

In one embodiment, the liquid compositions of the invention comprise at least one mannanase. “Mannanases” according to the invention are mannan degrading enzymes selected from β-mannosidase (EC 3.2.1.25), endo-1,4-β-mannosidase (EC 3.2.1.78), and 1,4-β-mannobiosidase (EC 3.2.1.100). Preferably, at least one mannan degrading enzyme is selected from the group of endo-1,4-β-mannosidase (EC 3.2.1.78), a group of enzymes which may be called endo-β-1,4-D-mannanase, β-mannanase, or mannanase herein.

Mannan degrading activity may be determined according to standard test procedures known in the art, such as using carob galactomannan dyed with Remazol Brilliant Blue as described in McCleary, B. V. (1978). Carbohydrate Research, 67(1), 213-221. Another method for testing mannan degrading activity uses detection of reducing sugars when incubated with substrate such as guar gum or locust bean gut—for reference see Miller, G. L. Use of Dinitrosalicylic Acid Reagent for Determination of Reducing Sugars. Analytical Chemistry 1959; 31: 426-428.

In one embodiment, the liquid compositions of the invention comprise at least one mannanase selected from alkaline mannanase of Family 5 or 26. The term “alkaline mannanase” is meant to encompass mannanases having an enzymatic activity at a given pH ranging from 7 to 12, preferably 7.5 to 10.5.

At least one mannanase comprised in the liquid compositions of the invention may be selected from GH5 family mannanase. In one embodiment, at least one mannanase originates from Bacillus clausii (Man6, SEQ ID NO:12, WO 2018/184767) or variants at least 90% identical thereto. In one embodiment, at least one mannanase originates from Bacillus hemicellulosilyticus (Man7, SEQ ID NO:16 of WO 2018/184767) and variants thereof, e.g. as disclosed in WO 2018/220274. In one embodiment, at least one mannanase originates from Virgibacillus soli (Man14, SEQ ID NO:20 of WO 2018/184767) and variants at least 90% identical thereto. Preferably, at least one mannanase has a polypeptide sequence 95% identical to a polypeptide sequence of SEQ ID NO:20 of WO 2018/184767, preferably having at least one substitution selected from A101V, E405G, and Y459F.

In one embodiment, at least one mannanase comprised in the liquid compositions of the invention is selected from WO 2009/074685 and variants at least 90% identical thereto.

In one embodiment, at least one mannanase comprised in the liquid compositions of the invention is selected from mannanases originating from Trichoderma organisms, such as disclosed in WO 93/24622 and WO 2008/009673.

In one embodiment, at least one mannanase comprised in the liquid compositions of the invention is selected from mannanases having a sequence according to positions 31-490 of SEQ ID NO:388 of WO 2005/003319 and variants which are preferably at least 90% identical thereto.

According to the present invention, the liquid compositions of the invention, in one embodiment, comprise a combination of at least two mannanases, preferably one of them being an alkaline mannanase; at least one mannanase is selected from the group of endo-1,4-β-mannosidase (EC 3.2.1.78) as disclosed above.

DNAse

In one embodiment, the liquid compositions of the invention comprise at least one DNAse. DNAses catalyzes the hydrolytic cleavage of phosphodiester linkages in DNA. The DNAses are classified e.g. in E.C. 3.1.11, E.C. 3.1.12, E.C. 3.1.15, E.C. 3.1.16, E.C. 3.1.21, E.C 3.1.22, E.C 3.1.23, E.C 3.1.24 and E.C.3.1.25 as well as EC 3.1.21.X, where X=1, 2, 3, 4, 5, 6, 7, 8 or 9.

DNAse activity can be determined by using the DNAseAlert™ Kit (11-02-01-04, IDT Integrated DNA Technologies) according to the supplier's manual. Briefly, 95 μl DNase sample is mixed with 5 μl substrate in a microtiter plate, and fluorescence is immediately measured using e.g. a Clariostar microtiter reader from BMG Labtech (536 nm excitation, 556 nm emission).

In one embodiment, at least one DNAse comprised in the liquid compositions of the invention is selected from polypeptides having an amino acid sequence which is at least 80% identical to SEQ ID NO: 1-24 and SEQ ID NO: 27-28 of WO 2019/081724 and WO 2019/081721. Variant DNAses comprises one or both of the motifs SEQ ID NO: 25 of WO 2019/081724 and SEQ ID NO: 26 of WO 2019/081724.

In one embodiment, at least one DNAse is selected from polypeptides comprising one or more of the motifs SEQ ID NO: 73 of WO 2017/060493, SEQ ID NO: 74 of WO 2017/060493 and SEQ ID NO: 75 of WO 2017/060493.

According to the present invention, the liquid compositions of the invention, in one embodiment, comprise a combination of at least two DNAses.

It is clear that the liquid formulation comprises further—besides the at least one amylase, the at least one cellulase, the at least one mannanase and/or the at least one DNAse—the components (a) and (b) and (c) and (d) being present in the liquid compositions in their ratios, amounts and total amounts as defined above in the chapters on component (a), component (b), component (c) and component (d).

Other Enzymes

In one embodiment, the liquid compositions of the invention comprise at least one enzyme selected from the group of lyases and dispersins.

In one embodiment, the liquid compositions of the invention comprise at least one lyase, preferably at least one carbon-oxygen lyase acting on polysaccharides. “Lyase” may be a pectate lyase (EC 4.2.2.2) derived from Bacillus, particularly B. licheniformis or B. agaradhaerens, or a variant derived of any of these, e.g. as described in U.S. Pat. No. 6,124,127, WO 99/027083, WO 99/027084, WO 2002/006442, WO 2002/092741, WO 2003/095638.

In one embodiment, the liquid compositions of the invention comprise at least one polypeptide having hexosaminidase activity (EC 3.2.1.52) like dispersins. Dispersins include but are not limited to dispersin B (DspB), which are usually described as β-N-acetylglucosamininidases belonging to the Glycoside Hydrolase 20 family (GH20). Preferably at least one dispersin comprised in liquid compositions of the invention is at least 80% identical to SEQ ID NO:10 as disclosed in WO 2017/186943.

Trialkylcitrate

In one embodiment, the liquid compositions of the invention comprise at least one compound according to formula (A):

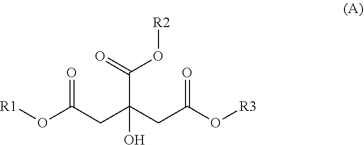

wherein the variables in formula (A) are defined as follows:
R1, R2, R3 are selected from H, linear C₃-C₈ alkyl, and branched C₃-C₈ alkyl, wherein at least one of R1, R2, and R3 is not H. Examples of linear C₁-C₈ alkyl are methyl, ethyl, n-propyl, n-butyl, n-pentyl, etc. Examples of branched C₃-C₈ alkyl are 2-propyl, 2-butyl, sec.-butyl, tert.-butyl, 2-pentyl, 3-pentyl, iso-pentyl, etc. Preferably, R1, R2, R3 are ethyl.

In one embodiment, the weight ratio of component (c):compound according to formula (A) is about 1:10, preferably about 1:6, more preferably about 1:4.

In one embodiment, liquid compositions comprise component (b) and a compound according to formula (A) in a weight ratio of 2:1 to 12:1.

Preferably, liquid compositions comprising at least one compound according to formula (A), comprise total amounts of component (b) ranging from about 40% to about 75% by weight, from about 50% to about 70% by weight, or from about 60% to about 70% by weight, all relating to the total weight of the liquid composition.

Preferably, component (b) comprises at least one “alpha-omega diol”, preferably selected from 1,6-hexane diol and diethylene glycol.

In one embodiment, liquid compositions of the invention are free from compounds according to formula (A).

Inventive liquid compositions usually are alkaline or exhibit a neutral or slightly acidic pH value, such as 5.0 to 10.0, 5.2 to 9.0, 5.4 to 8.0, or 5.6 to 7.0. In one embodiment, the liquid composition has a pH of about 6.

In one embodiment, the liquid compositions comprise amounts of water that add up the total weight of the liquid composition to 100%. Preferably, water is comprised in amounts in the range of 20% to 50% by weight, or 25% to 45% by weight, or 30% to 40% by weight, all relative to the total weight of the liquid composition.

Storage-Stability of Liquid Composition

In one embodiment, the liquid compositions comprising components (a), (b), optionally (c) and optionally (d) are homogenous at ambient temperature, and normal pressure of about 101.3 kPa. Homogenous means that the liquid compositions do not show visible particles or turbidity. Preferably, the liquid composition comprises at least components (a)-(c).

In one embodiment, the liquid compositions retain homogeneity during storage at a temperature of about 4° C., 30° C. and/or 37° C. Preferably, the liquid compositions retain homogeneity for a time period of at least up to 50 days or at least up to 200 days. “Retain homogeneity” equals “remains homogenous” herein.

Preferably, “storage-stability/storage-stable” refers to both homogeneity of the liquid composition and (sufficient) residual enzyme activity within such enzyme-containing liquid composition.

To determine changes in enzymatic activity over time, usually the “initial enzymatic activity” of an enzyme is measured under defined conditions at time zero (i.e. before storage) and the “enzymatic activity after storage” is measured at a certain point in time later (i.e. after storage).

The enzymatic activity after storage divided by the initial enzymatic activity multiplied by 100 gives the “residual enzymatic activity” (a %).

In one embodiment, liquid compositions of the invention are storage-stable relating to hydrolase activity, preferably proteolytic activity and/or lipolytic activity. Storage-stability of a hydrolase (component (c)) according to the invention relates to residual enzymatic activity of at least 60%, at least 70%, or at least 80% after storage of the liquid compositions for at least up to 28 days at elevated temperatures of about 30° C. or about 37° C. when compared to the initial enzymatic activity available before storage. In a preferred embodiment, at least one hydrolase is selected from hydrolases comprising as a catalytic triad the amino acids aspartate, histidine and serine, preferably proteases and lipases as disclosed above.

Preferably, the hydrolytic activity is proteolytic activity. More preferably, proteolytic activity relates to the activity of at least one subtilisin proteases (EC 3.4.21.62), preferably a protease according to SEQ ID NO:22 as described in EP 1921147 or variants thereof having proteolytic activity, preferably a protease 80% identical to SEQ ID NO:22 as described in EP 1921147 having at least R101E.

The invention relates to a method of stabilizing of at least one hydrolase as disclosed as component (c) by the step of adding component (a) and component (b) and optionally component (d) in one or more steps, wherein components (a) and (b) and (c) and (d) are those disclosed above, with their ratios, amounts and total amounts per liquid composition and their preferred ranges as also disclosed in detail before. Preferably, component (c) comprises at least one hydrolase comprising as a catalytic triad the amino acids aspartate, histidine and serine. Preferably, at least one hydrolase is selected from proteases and lipases as disclosed above.

Preferably component (c) comprises at least one subtilisin protease (EC 3.4.21.62), preferably a protease according to SEQ ID NO:22 as described in EP 1921147 or variants thereof having proteolytic activity, preferably a protease 80% identical to SEQ ID NO:22 as described in EP 1921147 having R101E.

Use of “Diol (Other than 1,2-Propane Diol)”

In one aspect, the invention relates to the use of at least one “diol (other than 1,2-propane diol)” selected from alpha-omega diols, 1,2 butane-diol, 1,3-butane diol, 1,2-pentane diol, and 1,2-hexane diol to remove turbidity and/or dissolve particles from a liquid composition comprising at least 1,2-propane diol and a tripeptide aldehyde according to formula (PA).

In one aspect, the invention relates to the use of “diol (other than 1,2-propane diol)” to increase the solubility of a peptide aldehyde as disclosed as component (a) herein, preferably at least one tripeptide aldehyde. “Increase of solubility” herein means, that turbidity and/or particles present in a liquid composition is reduced or removed by addition of at least one “diol (other than 1,2-propane diol)”. Preferably, increase of solubility of at least one peptide aldehyde according to the invention occurs when said peptide aldehyde is provided in 1,2-propane diol and component “diol (other than 1,2-propane diol)” is added.

In one embodiment, the addition of at least one “diol (other than 1,2-propane diol)” increases homogeneity of a liquid composition comprising at least 1,2-propane diol and at least one peptide aldehyde as disclosed herein when compared to homogeneity of a liquid composition lacking at least one “diol (other than 1,2-propane diol)”.

Preferably at least one peptide aldehyde is a tripeptide aldehyde according to formula (PA) characterized by R¹ and R² being groups such that NH—CHR¹—CO and/or NH—CHR²—CO are nonpolar amino acids, preferably selected from an L or D-amino acid residue of Ala, Val, Gly and Leu. R³ preferably is a group such that NH—CHR³—CO is an L or D-amino acid residue of Tyr, Phe, Val, Ala or Leu.

In one embodiment, R¹ is a group such that NH—CHR¹—CO is an L or D-amino acid residue of Gly or Val, R² is a group such that NH—CHR²—CO is an L or D-amino acid residue of Ala, and R³ is a group such that NH—CHR³—CO is an L or D-amino acid residue of Tyr, Ala, or Leu.

In one embodiment, at least two selected from R¹, R² and R³ are groups such that NH—CHR¹—CO and/or NH—CHR²—CO and/or NH—CHR³—CO are non-polar amino acid residues, preferably independently from each other selected from an L or D-amino acid residue of Ala, Val, Gly and Leu.

In one embodiment, R¹ is a group such that NH—CHR¹—CO is an L or D-amino acid residue of Val, R² is a group such that NH—CHR²—CO is an L or D-amino acid residue of Ala, and R³ is a group such that NH—CHR³—CO is an L or D-amino acid residue of Leu.

Preferably, at least one “diol (other than 1,2-propane diol)” is selected from alpha-omega diols 1,2 butane-diol, 1,3-butane diol, 1,2-pentane diol and 1,2-hexane diol. More preferably, at least one “diol (other than 1,2-propane diol)” is selected from “alpha-omega diols”, 1,2-pentane diol, and 1,2-hexane diol. “diol (other than 1,2-propane diol)” is as disclosed herein in the chapter relating to component (b).

“Alpha-omega diols” are preferably selected from 1,3-propane diol, 1,4-butane diol, 1,6-hexane diol, diethylene glycol and dipropylene glycol. Most preferably, at least one alpha-omega diol is selected from 1,6-hexane diol and diethylene glycol. Even more preferred is the use of 1,6-hexane diol.

The concentrations of the individual components of the liquid composition, their ratios, amounts and total amounts are as disclosed above.

In one embodiment, the use of at least one “diol (other than 1,2-propane diol)” as disclosed above results in increased homogeneity of the liquid compositions of the invention, when compared to liquid compositions lacking at least one “diol (other than 1,2-propane diol)”. Homogeneity, in one aspect relates to the product being a clear solution at a temperature of about 20° C., and normal pressure of about 101.3 kPa. “Clear solution” relates to homogeneity since no turbidity and/or visible particles are present.

Use of Solvent-System to Provide Storage-Stable Liquid Composition

In one aspect, the invention relates to the use of component (b) to dissolve at least one peptide aldehyde (component (a) as disclosed herein), preferably at least one tripeptide aldehyde, to provide a homogenous liquid composition, which preferably is storage stable.

In one embodiment, the invention relates to the use of component (b) as disclosed herein to provide a storage stable liquid composition comprising at least one peptide aldehyde (component (a) as disclosed herein) and at least one hydrolase (component (c) as disclosed herein).

In a preferred embodiment, at least one hydrolase (component (c)) is selected from hydrolases comprising as a catalytic triad the amino acids aspartate, histidine and serine, preferably proteases and lipases as disclosed above.

In one embodiment, at least one hydrolase (component (c)) is a subtilisin proteases (EC 3.4.21.62), preferably a protease according to SEQ ID NO:22 as described in EP 1921147 or variants thereof having proteolytic activity, preferably a protease 80% identical to SEQ ID NO:22 as described in EP 1921147 having R101E.

Preferably, “storage-stability/storage-stable” refers to both homogeneity of the liquid composition and sufficient residual enzyme activity within such enzyme-containing liquid composition.

Components (a) and (b) and (c) are as disclosed herein.

Preparation of Liquid Composition

The invention relates to a process for making the liquid compositions of the invention, said process comprising the step of mixing in one or more steps the components of the liquid compositions in any order.

In one embodiment, at least components (a) and (b) and optionally (c) and (d) are mixed with water in one or more steps in any order. All components are those disclosed above.

In one embodiment, at least one enzyme comprised in component (c) is part of a liquid enzyme concentrate.

In one embodiment, at least components (a) and (b) and optionally (c) and (d) are mixed together in one or more steps in any order, prior to addition of water.

In one embodiment, component (a) is mixed with component (b), before components (c) and (d) are added in one or more steps, prior to addition of water.

In one embodiment, component (b) is prepared by mixing 1,2-propanediol with at least one “diol (other than 1,2-propane diol)” in one or more steps before addition of components (a) and (c) and component (d) and water in one or more steps. Preferably, component (a) is mixed with component (b) before addition of components (c) and (d) and water. Preferably addition of water is the last step.

In one embodiment, part of component (b) is mixed with component (a) and the rest of component (b) is added together with component (c), before component (d) and water are added in one or more steps.

In one embodiment, at least components (a) and (b) are mixed at mixing temperatures up to 65° C., up to 60°, up to 55° C., or up to 50° C. Preferably, mixing temperatures are ranging from ambient temperature (meaning room temperature herein, preferably 15° C.-30° C.) to the temperature where of at least one “diol (other than 1,2-propane diol)” is liquid. For example, the mixing temperature ranges from ambient temperature to 65° C., ranging from ambient temperature to 60° C., ranging from ambient temperature to 55° C., ranging from ambient temperature to 50° C.

In one embodiment, the process for making the liquid compositions of the invention comprises the steps of
i) mixing component (a) with a part of 1,2-propanediol,
ii) mixing component (c) with a part of 1,2-propanediol,
iii) preparing component (b) by mixing a part of 1,2-propanediol with at least one “diol (other than 1,2-propane diol)”
iv) adding the mixture of step i) to the mixture of step iii)
v) adding the mixture of step ii) to the mixture of step iv)
vi) adding at least one water-soluble salt and water.

Preferably, the mixture of step iii) is prepared at a mixing temperature ranging from ambient temperature to 55° C. or ranging from 40° C. to 50° C. Alternatively, at least one “diol (other than 1,2-propane diol)” is heated above the melting point to liquify it before mixing it with 1,2-propane diol.

In one embodiment, the mixing steps i) and/or iv) are executed at ambient temperature.

In one embodiment, the mixing steps ii) and/or v) and/or vi) are executed at a temperature ranging from 4° C. to ambient temperature. Preferably, at least one enzyme comprised in component (c) is part of a liquid enzyme concentrate.

The mixing steps are preferably done under stirring. Stirring is done until the liquid composition is a clear solution (i.e. homogenous in visibility; homogeneity in this context means that the liquid composition does not show visible particles or turbidity).

Amounts of the individual components are as disclosed herein. Water is added up to a 100% by weight liquid composition.

Preferably pH is adjusted before water is added.

Components (a), (b), (c), and (d) and optionally further components are as disclosed herein.

Detergent Formulations

In one aspect the invention relates to a detergent formulation comprising
component (a): at least one peptide aldehyde, preferably a tripeptide aldehyde
component (b): a mixture of at least two organic solvents, wherein preferably at least one of the organic solvents is 1,2-propane diol
component (c): at least one enzyme selected from the group of hydrolases (EC 3), preferably comprising a catalytic triad having the motif serine-histidine-aspartate as disclosed herein,
and at least one detergent component.

In one embodiment, the detergent formulations are liquid. Liquid detergent formulations of the invention include solutions, emulsions and dispersions, gels etc. as long as the liquid is fluid and pourable. Liquid detergent formulations according to the present invention preferably have a dynamic viscosity in the range of about 500 to about 20,000 mPa*s, determined at 25° C. according to Brookfield, for example spindle 3 at 20 rpm with a Brookfield viscosimeter LVT-II.

In a preferred embodiment, at least one hydrolase (component (c)) comprised in the detergent formulation is selected from those with a catalytic triad having a motif selected from aspartate-histidine-serine and serine-histidine-aspartate, wherein the relative order of these amino acids reads from the amino to carboxy-terminus. In a preferred embodiment, the hydrolase has a catalytic triad with the motif serine-histidine-aspartate, wherein the relative order of these amino acids reads from the amino to carboxy-terminus. More preferably, said hydrolase is selected from proteases and lipases, as disclosed above.

In one embodiment, detergent formulations of the invention comprise the liquid composition of the invention and at least one detergent component, wherein component (c) in the liquid composition preferably comprises at least one protease and/or at least one lipase.

In another embodiment, additionally to at least one protease and/or at least one lipase, the detergent formulation additionally comprises at least one hydrolase selected from amylases, cellulases, mannanases, and DNAses, all as disclosed herein. The detergent formulation may also comprise at least one other enzyme useful in cleaning processes selected from lyases, preferably pectate lyases (EC 4.2.2.2) and dispersins.

The invention also relates to a method for preparation of a detergent formulation of the invention, comprising the steps of mixing at least components (a) and (b) and (c) and/or optionally (d) and/or at least one further hydrolase selected from amylases, cellulases, mannanases, and DNAses—all as disclosed herein—with at least one detergent in one or more steps in any order.

In one embodiment, the method of preparation a detergent formulation comprises the steps of mixing the liquid compositions of the invention with at least one detergent component in one or more steps in any order.

Components (a) and (b) and (c) and/or (d) and/or “further components” are such that their amounts in the liquid compositions comply with their ratios, amounts and total amounts as defined above in the chapters on component (a), component (b), component (c) and component (d), and thus the detergent formulation comprises those components in such amounts and ratios which result from the use of such liquid composition in such detergent formulation.

The invention in one aspect relates to the use of the liquid compositions of the invention as a detergent component for detergent formulations such as I&I and homecare formulations for laundry and hard surface cleaning.

“Detergent formulation” or “cleaning formulation” herein means formulations designated for cleaning soiled material. Cleaning preferably means laundering or hard surface cleaning. Soiled material according to the invention includes textiles and/or hard surfaces.

Detergent components vary in type and/or amount in a detergent formulation depending on the desired application such as laundering white textiles, colored textiles, and wool. The component(s) chosen further depend on physical form of a detergent formulation (liquid, solid, gel, provided in pouches or as a tablet, etc). The component(s) chosen e.g. for laundering formulations further depend on regional conventions which themselves are related to aspects like washing temperatures used, mechanics of laundry machine (vertical vs. horizontal axis machines), water consumption per wash cycle etc. and geographical characteristics like average hardness of water.

Individual detergent components and usage in detergent formulations are known to those skilled in the art. Suitable detergent components comprise inter alia surfactants, builders, polymers, alkaline, bleaching systems, fluorescent whitening agents, suds suppressors and stabilizers, hydrotropes, and corrosion inhibitors. Further examples are described e.g. in “complete Technology Book on Detergents with Formulations (Detergent Cake, Dishwashing Detergents, Liquid & Paste Detergents, Enzyme Detergents, Cleaning Powder & Spray Dried Washing Powder)”, Engineers India Research Institute (EIRI), 6^{th} edition (2015). Another reference book for those skilled in the art may be “Detergent Formulations Encyclopedia”, Solverchem Publications, 2016.

It is understood that the detergent components are in addition to the components comprised in the liquid compositions of the invention. If a component comprised in the liquid compositions of the invention is also a detergent component, it might be the concentrations that need to be adjusted that the component is effective for the purpose desired in the detergent formulation.

Detergent components may have more than one function in the final application of a detergent formulation, therefore any detergent component mentioned in the context of a specific function herein, may also have another function in the final application of a detergent formulation. The function of a specific detergent component in the final application of a detergent formulation usually depends on its amount within the detergent formulation, i.e. the effective amount of a detergent component.

The term “effective amount” includes amounts of individual components to provide effective stain removal and/or effective cleaning conditions (e.g. pH, quantity of foaming), amounts of certain components to effectively provide optical benefits (e.g. optical brightening, dye transfer inhibition), and/or amounts of certain components to effectively aid the processing (maintain physical characteristics during processing, storage and use; e.g. viscosity modifiers, hydrotropes, desiccants).

In one embodiment, a detergent formulation is a formulation of more than two detergent components, wherein at least one component is effective in stain-removal, at least one component is effective in providing the optimal cleaning conditions, and at least one component is effective in maintaining the physical characteristics of the detergent.

Detergent formulations of the invention in one aspect comprise the liquid compositions of the invention, wherein the liquid composition is physically isolated from detergent components.

In an embodiment the physical isolation occurs by using multi-compartment containers, preferably multi-compartment pouches. Such pouches are preferably formed by water-soluble polymeric films. Pouches can be of any form, shape and material which is suitable for holding a formulation, e.g., without allowing the release of said formulation from the pouch prior to water contact. The pouches may comprise a solid formulation and/or a liquid formulation in different compartments. The compartment for liquid components can be different in formulation than compartments containing solids (see e.g. EP 2014756).

In another embodiment, physical isolation occurs by microencapsulation. The aim of microencapsulation is, at the one hand, the isolation of the liquid core formulation from its surrounding, and, on the other hand, release of the core formulation at the time of use (the liquid core formulation must be released timely). Capsule contents usually are released by melting the wall, or dissolving it under particular conditions. In other systems, the wall is broken by solvent action, enzyme attack, chemical reaction, hydrolysis, or slow disintegration. Most prominently, the limiting factor for suitability in detergent formulations is a rapid release of the core formulation at the time when a detergent formulation is diluted in water but ensuring non-release of the core formulation during storage in detergent formulations. Microcapsules preferably are dispersed in liquid formulations with optional stabilization of such dispersions by means such as rheology modification through addition of thickeners. Stabilization of dispersions often is achieved by supplementation with dispersing agents. Microencapsulated liquid formulations may be part of a solid detergent formulation after drying of the microcapsules.

In one embodiment, at least a part of the detergent formulation of the invention is provided as a liquid. Depending on whether a water-soluble package is enclosing the liquid detergent formulation, the liquid detergent formulation comprises water or is essentially free from water.

Detergent formulations of the invention comprise at least one compound selected from surfactants, builders, polymers, fragrances and dyestuffs.

The detergent formulations of the invention comprise at least one surfactant selected from nonionic surfactants, amphoteric surfactants, anionic surfactants, and cationic surfactants.

The detergent formulations, in one embodiment, comprise 2% to 30% by weight of anionic surfactants and/or 2% to 30% by weight of non-ionic surfactants, all relative to the total weight of the detergent formulation. Usually, laundry detergents comprise higher amounts of surfactants than detergents for automated dish washing.

In one embodiment, the detergent formulations of the invention comprise at least one non-ionic surfactant selected from alkoxylated alcohols, di- and multiblock copolymers of ethylene oxide and propylene oxide and reaction products of sorbitan with ethylene oxide or propylene oxide, alkyl polyglycosides (APG), hydroxyalkyl mixed ethers and amine oxides.

In one embodiment, the detergent formulations of the invention comprise at least one non-ionic surfactant of the general formula (NIS-I):

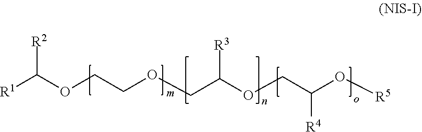

wherein in formula (NIS-I) the following applies:
R¹ is selected from C₁-C₂₃ alkyl and C₂-C₂₃ alkenyl, wherein alkyl and/or alkenyl are linear (straight-chain; n-) or branched; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.
R² is selected from H, C₁-C₂₀ alkyl and C₂-C₂₀ alkenyl, wherein alkyl and/or alkenyl are linear (straight-chain; n-) or branched.
R³ and R⁴, each independently selected from C₁-C₁₆ alkyl, wherein alkyl is linear (straight-chain; n-) or branched; examples are methyl, ethyl, n-propyl, isopropyl, n-butyl, isobutyl, sec-butyl, tertbutyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.
R⁵ is selected from H and C₁-C₁₈ alkyl, wherein alkyl is linear (straight-chain; n-) or branched.

The integers of the general formula (NIS-I) are defined as follows:
m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

The non-ionic surfactants of the general formula (NIS-I) can be of any structure, is it block or random structure, and is not limited to the displayed sequence of formula (NIS-I).

In a preferred embodiment, the detergent formulations comprise at least one non-ionic surfactant selected from compounds of general formula (NIS-I), wherein said non-ionic surfactant is characterized in R¹ being n-C₁₃H₂₇, R² and R⁵ being H, m being 3-20, n and o=0.

In a preferred embodiment, the detergent formulations comprise at least one non-ionic surfactant selected from compounds of general formula (NIS-I), wherein said non-ionic surfactant is characterized in R¹ being linear or branched C₁₀ alkyl, R² and R⁵ being H, m being 3-14, n and o=0.

In a preferred embodiment, the detergent formulations comprise at least two non-ionic surfactants, selected from compounds of general formula (NIS-I), wherein one of said non-ionic surfactants is characterized in R¹ being n-C₁₅H₃₁, R² and R⁵ being H, m being 11-80, n and o=0, and the other surfactant is characterized in R¹ being n-C₁₇H₃₅, R² and R⁵ being H, m being 11-80, n and o=0.

In a preferred embodiment, the detergent formulations comprise at least two non-ionic surfactants, selected from compounds of general formula (NIS-I), wherein one of said non-ionic surfactants is characterized in R¹ being n-C₁₂H₂₅, R² and R⁵ being H, m being 3-30, preferably 7, n and o=0, and the other surfactant is characterized in R¹ being n-C₁₄H₂₉, R² and R⁵ being H, m being 3-30, preferably 7, n and o=0.

In a preferred embodiment, the detergent formulations comprise at least two non-ionic surfactants, selected from compounds of general formula (NIS-I), wherein one of said non-ionic surfactants is characterized in R¹ being n-C₁₁H₂₃, R² and R⁵ being H, m being 4-10, n and o=0, and the other surfactant is characterized in R¹ selected from n-C₁₁H₂₃ and n-C₁₇H₃₅, R² and R⁵ being H, m being 4-10, n and o=0.

In a preferred embodiment, the detergent formulations comprise at least two non-ionic surfactants, selected from compounds of general formula (NIS-I), wherein one of said non-ionic surfactants is characterized in R¹ being n-C₉H₁₉, R² and R⁵ being H, m being 5-7, n and o=0, and the other surfactant is characterized in R¹ being n-C₁₇H₃₅, R² and R⁵ being H, m being 5-7, n and o=0.

In a preferred embodiment, the detergent formulations comprise at least two non-ionic surfactants, selected from compounds of general formula (NIS-I), wherein one of said non-ionic surfactants is characterized in R¹ being n-C₁₁H₂₃, R⁵ being H, m is 7, n and o=0, and the other surfactant is characterized in R¹ being C₁₃H₂₇, R⁵ being H, m being 7, n and o=0.

In one embodiment, detergent formulations comprising at least one non-ionic surfactant according to formula (NIS-I), preferably those as disclosed above, are laundry detergents.

In one embodiment, the detergent formulations of the invention comprise at least one non-ionic surfactant of the general formula (NIS-II):

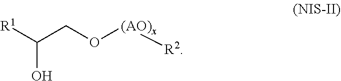

wherein in formula (NIS-II) the following applies:
AO being identical or different alkylene oxides, selected from CH₂—CH₂—O, (CH2)3-O, (CH₂)₄—O, CH₂CH(CH₃)—O, CH(CH₃)—CH₂—O— and CH₂CH(n-C₃H₇)—O.
R¹ is selected from linear (straight-chain; n-) or branched C₄-C₃₀-alkyl, and from straight-chain or branched C₄-C₃₀-alkylene with at least one C—C double bond. R¹ preferably is selected from straight-chain or branched C₄-C₃₀-alkyl, n-C₄-C₃₀-alkyl, n-C₇-C₁₅ alkyl, or n-C₁₀-C₁₂-alkyl.
R² is selected from linear (straight-chain; n-) or branched C₁-C₃₀-alkyl, and from straight-chain or branched C₂-C₃₀-alkylene with at least one C—C double bond. R² preferably is selected from straight-chain or branched C₆-C₂₀-alkyl, preferably straight-chain or branched C₆-C₁₂-alkyl, more preferably straight-chain or branched C₁₀-C₁₂-alkyl.

The integer x of the general formula (NIS-II) preferably is a number in the range of 5 to 70, 10 to 60, 15 to 50, or 20 to 40.

In a preferred embodiment, the detergent formulations of the invention comprise at least one non-ionic surfactant according to formula (NIS-II), wherein R¹ is n-C₃-C₁₇ alkyl, R² is linear or branched C₈-C₁₄ alkyl. Preferably AO is selected from —(CH₂CH₂O)ₓ₂—(CH₂CH(CH₃)—O)ₓ₃, —(CH₂CH₂O)ₓ₂—(CH(CH₃)CH₂—O)ₓ₃, and —(CH₂CH₂O)ₓ₄, wherein x2 and x4 is a number in the range of 15-50 and x3 is a number in the range of 1 to 15.

In a preferred embodiment, the detergent formulations of the invention comprise at least one non-ionic surfactant according to formula (NIS-II), wherein R¹ is n-C₈ alkyl, R² is branched C₁₁ alkyl, AO is CH₂—CH₂—O, and x is 22.

In a preferred embodiment, the detergent formulations of the invention comprise at least one non-ionic surfactant according to formula (NIS-II), wherein R¹ is n-C₈ alkyl, R² is n-C₅-C₁₀ alkyl, AO is CH₂—CH₂—O, and x is 40.

In a preferred embodiment, the detergent formulations of the invention comprise at least one non-ionic surfactant according to formula (NIS-II), wherein R¹ is n-C₈ alkyl, R² is n-C₁₀ alkyl, AO is selected from —(CH₂CH₂O)ₓ₂—(CH₂CH(CH₃)—O)₃, —(CH₂CH₂O)ₓ₂—(CH(CH₃)CH₂—O)ₓ₃, wherein x2 is 22 and x3 is 1.

In one embodiment, detergent formulations comprising at least one non-ionic surfactant according to formula (NIS-II), preferably those as disclosed above, are automated dishwashing detergents. Preferably, the automated dishwashing detergents comprise at least one compound according to formula (NIS-II) in the range of about 0.3% to 10% by weight, in the range of about 0.5% to 5% by weight, or in the range of about 1% to 3%, all relative to the total weight of a detergent formulation. In one embodiment, at least one non-ionic surfactant is a compound according to formula (NIS-II), wherein R¹ is n-C₈ alkyl, R² is branched C₁₁ alkyl, AO is CH₂—CH₂—O, and x is 22.

In one embodiment, the detergent formulations of the invention comprise at least one anionic surfactant selected from alkali metal and ammonium salts of C₃-C₁₈-alkyl sulfates, of C₃-C₁₈-fatty alcohol polyether sulfates, of sulfuric acid half-esters of ethoxylated C₈-C₁₂-alkylphenols (ethoxylation: 1 to 50 mol of ethylene oxide/mol), C₁₂-C₁₈ sulfo fatty acid alkyl esters, for example of C₁₂-C₁₈ sulfo fatty acid methyl esters, furthermore of C₁₂-C₁₈-alkylsulfonic acids and of C₁₀-C₁₈-alkylarylsulfonic acids. Preference is given to the alkali metal salts of the aforementioned compounds, particularly preferably the sodium salts. Non-limiting examples of further suitable anionic surfactants include branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, secondary alkanesulfonates (SAS), paraffin sulfonates (PS), sulfonated fatty acid glycerol esters, alkyl- or alkenylsuccinic acid, fatty acid derivatives of amino acids, diesters and monoesters of sulfo-succinic acid.

In one embodiment, the detergent formulations of the invention comprise at least one anionic surfactant of general formula (AS-I):

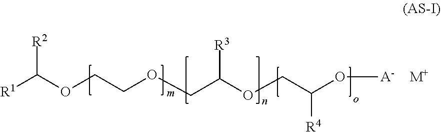

Wherein in formula (AS-I) the following applies:
R¹ is selected from C₁-C₂₃-alkyl (such as 1-, 2-, 3-, 4-C₁-C₂₃-alkyl) and C₂-C₂₃-alkenyl, wherein alkyl and/or alkenyl are linear (straight-chain; n-) or branched, and wherein 2-, 3-, or 4-alkyl; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.
R² is selected from H, C₁-C₂₀-alkyl and C₂-C₂₀-alkenyl, wherein alkyl and/or alkenyl are linear (straight-chain; n-) or branched.
R³ and R⁴, each independently selected from C₁-C₁₆-alkyl, wherein alkyl is linear (straight-chain; n-) or branched; examples are methyl, ethyl, n-propyl, isopropyl, n-butyl, isobutyl, sec-butyl, tertbutyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.
A⁻ is selected from —RCOO⁻, —SO₃⁻ and RSO₃⁻, wherein R is selected from linear (straight-chain; n-) or branched C₁-C₈-alkyl, and C₁-C₄ hydroxyalkyl, wherein alkyl is. Compounds might be called (fatty) alcohol/alkyl (ethoxy/ether) sulfates [(F)A(E)S] when A⁻ is SO₃⁻, (fatty) alcohol/alkyl (ethoxy/ether) carboxylat [(F)A(E)C] when A⁻ is —RCOO⁻.
M⁺ is selected from H and salt forming cations. Salt forming cations usually are monovalent or multivalent; hence M⁺ equals 1/v M^{v+}. Examples include but are not limited to sodium, potassium, magnesium, calcium, ammonium, and the ammonium salt of mono-, di, and triethanolamine.

The integers of the general formula (AS-I) are defined as follows:
m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

Anionic surfactants of the general formula (AS-I) can be of any structure, block copolymers or random copolymers.

In a preferred embodiment, the detergent formulations of the invention comprise at least one anionic surfactant according to formula (AS-I), wherein R¹ is n-C₁₁H₂₃, R² is H, A⁻ is SO₃⁻, m, n and o being 0. M⁺ preferably is NH₄⁺. Such compounds may be called ammonium lauryl sulfate (ALS) herein.

In a preferred embodiment, the detergent formulations of the invention comprise at least one anionic surfactant according to formula (AS-I), wherein R¹ is n-C₁₁H₂₃, R² is selected from H, A⁻ is SO₃⁻, m being 2-5, preferably 3, and n and o being 0. M⁺ preferably is Na⁺. Such compounds may be called laurylethersulfates (LES) herein, preferably sodium laurylethersulfates (SLES).

The detergent formulations, in one embodiment, comprise at least two anionic surfactants, selected from compounds of general formula (AS-I), wherein one of said anionic surfactants is characterized in R¹ being C₁₁, R² being H, m being 2, n and o=0, A⁻ being SO₃⁻, M⁺ being Na⁺ and the other surfactant is characterized in R¹ being C₁₃, R² being H, m being 2, n and o=0, A⁻ being SO₃⁻, M⁺ being Na⁺.

Typically, laundry detergent formulations comprise compounds according to formula AS-I.

In one embodiment, the detergent formulations comprise at least one anionic surfactant selected from compounds of general formula (AS-II):

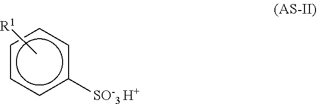

wherein R¹ in formula (AS-II) is C₁₀-C₁₃ alkyl. Detergent formulations of the invention, in one embodiment, comprise salts of compounds according to formula (AS-II), preferably sodium salts.

In a preferred embodiment, the detergent formulations of the invention comprise at least two anionic surfactants, selected from compounds of general formula (AS-II), wherein one of said anionic surfactants is characterized in R¹ being C₁₀, and the other surfactant is characterized in R¹ being C₁₃. Compounds like this may be called LAS (linear alkylbenzene sulfonates) herein.

Typically, laundry detergent formulations comprise compounds according to formula AS-II.

In one embodiment, detergent formulations comprise at least one anionic surfactant selected from AS-I and AS-II and at least one soap. In one embodiment, soaps are selected from salts (M⁺) of saturated and unsaturated C₁₂-C₁₃ fatty acids, such as lauric acid, myristic acid, palmitic acid, stearic acid, behenic acid, oleic acid, (hydrated) erucic acid. M⁺ is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine. Further non-limiting examples of suitable soaps include soap mixtures derived from natural fatty acids such as tallow, coconut oil, palm kernel oil, laurel oil, olive oil, or canola oil. Such soap mixtures comprise soaps of lauric acid and/or myristic acid and/or palmitic acid and/or stearic acid and/or oleic acid and/or linoleic acid in different amounts, depending on the natural fatty acids from which the soaps are derived.

In one embodiment, the detergent formulations of the invention comprise at least one amphoteric surfactant according to general formula (AMS-I), which might be called modified amino acids (proteinogenic as well as non-proteinogenic):

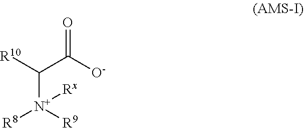

The variables in general formula (AMS-I) are defined as follows:
R⁸ is selected from H, C₁-C₄ alkyl, C₂-C₄ alkenyl, wherein alkyl and/or are linear (straight-chain; n-) or branched.
R⁹ is selected from C₁-C₂₂-alkyl, C₂-C₂₂-alkenyl, C₁₀-C₂₂ alkylcarbonyl, and C₁₀-C₂₂ alkenylcarbonyl.
R¹⁰ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)methyl, isopropyl, —(CH₂)₂SCH₃, and —CH₂SH.
R^{x} is selected from H and C₁-C₄-alkyl.

In one embodiment, the detergent formulations of the invention comprise at least one amphoteric surfactant of general formulae (AMS-IIa), (AMS-IIb), or (AMS-IIc), which might be called betaines and/or sulfobetaines:

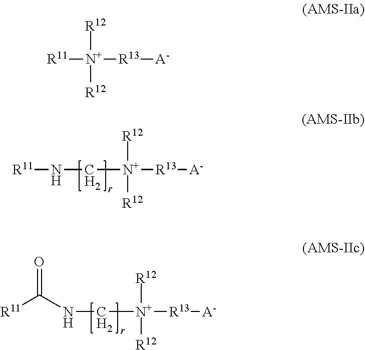

The variables in general formulae (AMS-IIa), (AMS-IIb) and (AMS-IIc) are defined as follows:
R¹¹ is selected from linear (straight-chain; n-) or branched C₇-C₂₂ alkyl and linear (straight-chain; n-) or branched C₇-C₂₂ alkenyl.
R¹² are each independently selected from linear (straight-chain; n-) C₁-C₄ alkyl.
R¹³ is selected from C₁-C₅ alkyl and hydroxy C₁-C₅ alkyl; for example 2-hydroxypropyl.
A⁻ is selected from carboxylate and sulfonate.

The integer r in general formulae (AMS-IIa), (AMS-IIb), and (AMS-IIc) is in the range of 2 to 6.

In one embodiment, the detergent formulations of the invention comprise at least one amphoteric surfactant of general formula (AMS-III), which might be called alkyl-amphocarboxylates:

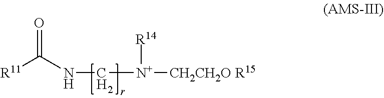

The variables in general formula (AMS-III) are defined as follows:
R¹¹ is selected from C₇-C₂₂ alkyl and C₇-C₂₂ alkenyl, wherein alkyl and/or alkenyl are linear (straight-chain; n-) or branched, preferably linear.
R¹⁴ is selected from —CH₂C(O)O⁻M⁺, —CH₂CH₂C(O)O⁻M⁺ and —CH₂CH(OH)CH₂SO₃⁻M⁺.
R¹⁵ is selected from H and —CH₂C(O)O⁻

The integer r in general formula (AMS-III) is in the range of 2 to 6.

Non-limiting examples of further suitable alkyl-amphocarboxylates include sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, and disodium capryloamphodipropionate.

In one embodiment, the detergent formulations of the invention comprise at least one amphoteric surfactant according to general formula (AMS-IV), which might be called amine oxides (AO):

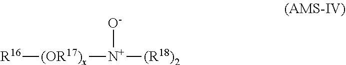

The variables in general formula (AMS-IV) are defined as follows:
R¹⁶ is selected from C₈-C₁₈ alkyl, hydroxy C₈-C₁₈ alkyl, acylamidopropoyl and C₈-C₁₈ alkyl phenyl group; wherein alkyl and/or alkenyl are linear (straight-chain; n-) or branched.
R¹⁷ is selected from C₂-C₃ alkylene, hydroxy C₂-C₃ alkylene, and mixtures thereof.
R¹⁸: each residue can be independently selected from C₁-C₃ alkyl and hydroxy C₁-C₃; R¹⁵ groups can be attached to each other, e.g., through an oxygen or nitrogen atom, to form a ring structure.

The integer x in general formula (AMS-IV) is in the range of 0 to 5, preferably from 0 to 3, most preferably 0.

Non-limiting examples of further suitable amine oxides include C₁₀-C₁₈ alkyl dimethyl amine oxides and C₈-C₁₈ alkoxy ethyl dihydroxyethyl amine oxides. Examples of such materials include dimethyloctyl amine oxide, diethyldecyl amine oxide, bis-(2-hydroxyethyl)dodecyl amine oxide, dimethyldodecylamine oxide, dipropyltetradecyl amine oxide, methylethylhexadecyl amine oxide, dodecylamidopropyl dimethyl amine oxide, cetyl dimethyl amine oxide, stearyl dimethyl amine oxide, tallow dimethyl amine oxide and dimethyl-2-hydroxyoctadecyl amine oxide.

A further example of a suitable amine oxide is cocamidylpropyl dimethylaminoxide, sometimes also called cocamidopropylamine oxide.

Mixtures of two or more different amphoteric surfactants, in one embodiment, are comprised in detergent formulations according to the present invention. Usually manual dishwashing detergents comprise at least one amphoteric surfactant.

In one embodiment, liquid detergent formulations according to the invention comprise at least one amphoteric surfactant, wherein the total amount of amphoteric surfactant preferably is in the range from 0.01% to 10%, in the range from 0.1 to 5%, or in the range from 0.5 to 1% by weight, all relative to the total weight of the detergent formulation. Preferably, at least one amphoteric surfactant is selected from compound according to formulae AMS-IIa, AMS-IIb, AMS-IIIc and AMS-IV.

In one embodiment, detergent formulations of the invention comprise one or more complexing agents (chelating agents, sequestrating agents), precipitating agents, and/or ion exchange compounds, which usually form water-soluble complexes with calcium and magnesium. Such compounds may be called “builders” or “building agents” herein, without meaning to limit such compounds to this function in the final application of a detergent formulation.

Non-phosphate based builders according to the invention include sodium gluconate, citrate(s), silicate(s), carbonate(s), phosphonate(s), amino carboxylate(s), polycarboxylate(s), polysulfonate(s), and polyphosphonate(s).

In a preferred embodiment, detergent formulations of the invention comprise one or more citrates. The term “citrate(s)” includes the mono- and the dialkali metal salts and in particular the mono- and preferably the trisodium salt of citric acid, ammonium or substituted ammonium salts of citric acid as well as citric acid as such. Citrate can be used as the anhydrous compound or as the hydrate, for example as sodium citrate dihydrate. The citric acid, in one embodiment, is provided as a mixture with formiate, e.g. Na-citrate:Na-formiate=9:1.

The detergent formulations of the invention, in one embodiment, comprise at least one phosphonate preferably selected from derivatives polyphosphonic acids such as of diphosphonic acid such as sodium salt of HEDP, derivatives of aminopolyphosphonic acid such as aminoalkylene phosphonic acids such as DTPMP. Said phosphonates preferably are comprised in amounts in the range of 0.1% to 5.0% by weight, in the range of 0.5% to 3.0% by weight, or in the range of 1.0% to 2.0% by weight, all relative to the total weight of the detergent formulation.

The detergent formulations of the invention, in one embodiment, comprise at least one aminocarboxylates, preferably selected from ethylenediaminetetraacetic acid (EDTA), diethylenetriaminepentaacetic acid (DTPA), methylglycine diacetate (MGDA), and glutamic acid diacetate (GLDA), which all may be (partially) neutralized with alkali. Said aminocarboxylates preferably are comprised in amounts in the range of 0% to 30.0% by weight, in the range of 0.5% to 25.0% by weight, in the range of 1% to 20% by weight, in the range of 2% to 15%, in the range of 2.5% to 10% by weight, in the range of 3% to 8% by weight, or in the range of 2.5 to 5% by weight, all relative to the total weight of the detergent formulation.

In one embodiment of the present invention, the formulations according to the invention are free from phosphates and polyphosphates, with hydrogenphosphates being subsumed, for example free from trisodiumphosphate, pentasodiumtripolyphosphate and hexasodiummetaphosphate. In connection with phosphates and polyphosphates, in the context of the present invention, “free from” is to be understood as meaning that the content of phosphate and polyphosphate is in total in the range from 10 ppm to 0.2% by weight, determined by gravimetry and relative to the total weight of the detergent formulation.

In one embodiment of the present invention, liquid detergent formulations comprise one or more viscosity modifiers. Depending on the viscosity desired, liquid detergent formulations of the invention usually comprise one or more rheology modifiers, which are also known as thickeners in the art. In one embodiment, the detergent formulation of the invention comprises at least one naturally derived polymeric structurants, preferably selected from polysaccharide derivatives such as xanthan gum in amounts in the range of 0.1% to about 5% by weight, or even from about 0.2% to about 0.5% by weight, relative to the total weight of the detergent formulation.

In one embodiment, liquid detergent formulations of the invention are free from bleaches, for example free from inorganic peroxide compounds or chlorine bleaches such as sodium hypochlorite, meaning that liquid detergent formulations according to the invention comprise in total 0.8%, 0.5%, 0.1% or 0.01% by weight or less of inorganic peroxide compound and chlorine bleach, relative in each case on total weight of the liquid detergent formulation.

The addition of the liquid compositions of the invention to a detergent formulation, preferably liquid detergent formulation, usually occurs in a weight ratio liquid composition:detergent formulation of about 1:1000, 1:500, 1:100, 1:50, 1:30, 1:25, 1:20, or 1:10.

Liquid detergent formulations therefore comprise different amounts of components (a), (b) and (c) of the liquid compositions, for example those listed in the table below (by weight means relative to the total weight of the liquid detergent):

| | Component (b) | | |
|---|---|---|---|
| | | Diol (other | |
| Component (a) | MPG | than MPG) | Component (c) |
| % by weight | % by weight | % by weight | % by weight |
| 0.0001-0.0005 | 0.045-0.055 | 0.002-0.01 | 0.004-0.0065 |
| 0.0002-0.001 | 0.09-0.11 | 0.004-0.02 | 0.008-0.013 |
| 0.001-0.005 | 0.45-0.55 | 0.02-0.1 | 0.04-0.065 |
| 0.002-0.01 | 0.9-1.1 | 0.04-0.2 | 0.08-0.13 |
| 0.003-0.02 | 1.5-1.83 | 0.07-0.33 | 0.13-0.22 |
| 0.004-0.02 | 1.8-2.2 | 0.08-0.4 | 0.16-0.26 |
| 0.005-0.015 | 2.25-2.75 | 0.1-0.5 | 0.2-0.325 |
| 0.01-0.05 | 4.5-5.5 | 0.4-1.0 | 0.4-0.065 |

The “diol (other than 1,2-propane diol)” is as disclosed in chapter relating to component (b).

Preferably, component (c) comprises at least one hydrolase comprising as a catalytic triad the amino acids aspartate, histidine and serine. Preferably, at least one hydrolase is a subtilisin protease (EC 3.4.21.62), preferably a protease according to SEQ ID NO:22 as described in EP 1921147 or variants thereof having proteolytic activity, preferably a protease 80% identical to SEQ ID NO:22 as described in EP 1921147 having R101E.

The total weight of 1,2-propane diol (MPG) in liquid detergent formulations, preferably those comprised in a container made of water-soluble polymeric film, may be added up to 35% by weight, relative to the total weight of the detergent formulation. “Added up” in this context means that additionally to the MPG comprised in the liquid composition of the invention, MPG is added up to the values as disclosed here in the final detergent formulation. Preferably, the total weight of MPG in liquid laundry detergents is added up to 30% by weight, up to 25% by weight, up to 20% by weight, up to 15% by weight, up to 10% by weight, up to 8% by weight, up to 7% by weight, or up to 6% by weight. The total amount of 1,2-propane diol in liquid detergent formulations preferably ranges from about 0.05% to 30% by weight, about 0.5% to 20% by weight, about 1% to 10% by weight, from about 2% to 8% by weight, from about 3% to 7% by weight, or from about 4% to 6% by weight, all relative to the total weight of the liquid detergent formulation.

In one embodiment, liquid detergent formulations of the invention comprise at least one solvent according to formula (A) as disclosed above, preferably triethyl citrate. In one embodiment, the total weight of triethyl citrate in liquid detergent formulations is added up to 3% by weight. Preferably, the total amount of triethyl citrate in the liquid detergent formulations ranges from about 0.0025% to 2.5% by weight. More preferably, the liquid detergent formulations comprise up to 1% by weight triethyl citrate. By weight means here relative to the total weight of the liquid detergent formulations.

The liquid detergent formulations comprising the components of the liquid compositions of the invention, are preferably selected from liquid laundry detergents, liquid manual dishwashing detergents and liquid automated dishwashing detergents.

In one embodiment, detergent formulations comprising the components of the liquid compositions of the invention are liquid laundry detergents. Usually, laundry detergents comprise relatively high amounts of surfactants, preferably selected from at least one non-ionic surfactant according to formula (NIS-I), and/or at least one anionic surfactant according to formula (AS-I) and/or at least one anionic surfactant according to formula (AS-II). Preferably, liquid laundry detergents of the invention comprise at least one non-ionic surfactant according to formula (NIS-I) and at least one anionic surfactant according to formula (AS-I) and at least one anionic surfactant according to formula (AS-II). In one embodiment, liquid laundry detergents comprise 2% to 25% by weight anionic surfactants, preferably 4% to 12% by weight, all % by weight relative to the total weight of the detergent formulation. In one embodiment, liquid laundry detergent formulations comprise 2% to 15% by weight non-ionic surfactants, preferably 3% to 10% by weight, all % by weight relative to the total weight of the detergent formulation.

The liquid compositions of the invention are preferably comprised in liquid laundry detergents in a weight ratio liquid composition:detergent of about 1:1000, 1:500, 1:100, 1:50, 1:30, 1:25, 1:20, or 1:10.

In one embodiment, liquid laundry detergents of the invention comprise at least one hydrolase (component (c), preferably comprising a catalytic triad having the motif serine-histidine-aspartate as disclosed herein) as disclosed herein. Preferably, at least one protease as disclosed herein is combined with one or more detergent components by addition of the liquid composition of the invention. Preferably, at least one protease is comprised in amounts of about 0.005% to 0.15% by weight, more preferably about 0.01% to 0.1% by weight, all relative to the total weight of the detergent formulation.

In one embodiment, liquid laundry detergents additionally comprise at least one lipase as disclosed herein, preferably in amounts of about 0.001% to 0.005%, more preferably 0.001% to 0.002% by weight, all relative to the total weight of the detergent formulation. AT least one lipase is selected from fungal triacylglycerol lipase is selected from Thermomyces lanuginosa lipase and variants thereof as disclosed herein.

In one embodiment, liquid laundry detergents additionally comprise at least one alpha-amylase as disclosed herein, preferably in amounts of about 0.001% to 0.015%, more preferably 0.002% to 0.015% by weight, all relative to the total weight of the detergent formulation.

In one embodiment, liquid laundry detergents additionally comprise at least one cellulase as disclosed herein, preferably in amounts of about 0.001% to 0.01%, more preferably 0.002% to 0.009% by weight, all relative to the total weight of the detergent formulation. At least one cellulase is selected from endoglucanases (EC 3.2.1.4), preferably those having the amino acid sequence disclosed in FIG. 14A-E of WO 91/17244 and variants thereof as disclosed herein.

In one embodiment, liquid laundry detergents additionally comprise at least one mannanase as disclosed herein, preferably in amounts of about 0.0005% to 0.005%, more preferably 0.0005% to 0.002% by weight, all relative to the total weight of the detergent formulation. At least one mannanase is selected from endo-1,4-β-mannosidase (EC 3.2.1.78) as disclosed herein.

In one embodiment, liquid laundry detergents comprise at least one aminocarboxylate selected from ethylenediaminetetraacetic acid (EDTA), diethylenetriaminepentaacetic acid (DTPA), methylglycine diacetate (MGDA), and glutamic acid diacetate (GLDA), all as disclosed above in amounts in the range of 1% to 10% by weight, in the range of 1.5% to 7% by weight, or in the range of 2% to 5% by weight all relative to the total weight of the liquid laundry detergent.

In one embodiment, liquid laundry detergents comprise citric acid in amounts in the range of 0.1% to 10% by weight, in the range of 0.5% to 8% by weight, in the range of 1% to 5% by weight, or in the range of 2% to 4% by weight, all relative to the total weight of the detergent formulation; the citric acid in one embodiment is provided as a mixture with formate, e.g. Na-citrate:Na-formate=9:1.

In one embodiment, liquid laundry detergents comprise at least one phosphonate as disclosed herein, preferably selected from HEDP and DTPMP, in amounts in the range of 0.5% to 3.0% by weight, or in the range of 1.0% to 2.5% by weight, all relative to the total weight of the detergent formulation.

In one embodiment, liquid laundry detergents comprise at least one ethoxylated polyethylenimine polymer which is based on a polyethylene core and a polyethylene oxide shell. Preferably, polyethylene imine core molecules are polyethylene imines with average molecular weight M_{w} in the range of 500 to 5000 g/mol. More preferred is a molecular weight from 500 to 1000 g/mol, even more preferred is an M_{w} of 600-800 g/mol. The ethoxylated polymer has in average 5 to 50, preferably 10 to 30 and even more preferably 15 to 25 EO (ethoxylate) groups per —NH group, resulting in an average molecular weight M_{w} in the range from 5,000 to 20,0000, preferably 8,000 to 100,000, more preferably 8,000 to 50,000, even more preferably 10,000 to 30,000, and most preferably 10,000 to 20,000) g/mol. Preferably at least one ethoxylated polyethyleneimine polymer is comprised in amounts ranging from about 0.5% to 5% by weight, from about 1% to 4%, or from about 1.5% to 3% by weight, all relative to the total weight of the liquid laundry detergent.

In one embodiment, liquid laundry detergents comprise at least one ethoxylated hexamethylene diamine polymer, which is preferably quaternized and—optionally but preferably-sulfated, preferably with an average molecular weight Mw in the range from 2000 to 10,000 g/mol, more preferably 3,000-8,000, most preferably 4,000-6,000. Preferably, ethoxylated hexamethylene diamine polymer, which is quaternized and—optionally but preferably-sulfated, contains in average 10 to 50, preferably 15 to 40 and even more preferably 20 to 30 EO (ethoxylate) groups per —NH group, resulting in an average molecular weight M_{w} in the range from 2,000 to 10,000 g/mol, more preferably 3,000-8,000, most preferably 4,000-6,000. In a preferred embodiment the ethoxylated hexamethylene diamine is quaternized and also sulfated, preferably bearing 2 cationic ammonium groups and 2 anionic sulfate groups. Preferably at least one ethoxylated hexamethylene diamine polymer is comprised in amounts ranging from about 0.5% to 5% by weight, from about 1% to 4%, or from about 1.5% to 3% by weight, all relative to the total weight of the liquid laundry detergent.

In one embodiment, detergent formulations comprising the components of the liquid compositions of the invention are liquid manual dishwashing detergents. The liquid compositions of the invention are preferably comprised in liquid manual dishwashing detergents in a weight ratio liquid composition:detergent of about 1:500, or 1:100.

Liquid manual dishwashing detergents preferably comprises at least one surfactant selected from ethoxylated or propoxylated sorbitan esters, amine oxides or alkyl polyglycosides, especially linear C₄-C₁₈-alkyl polyglucosides and branched C₈-C₁₈-alkyl polyglycosides such as compounds of general average formula (APG) are likewise suitable. APGs based on Guerbet alcohols are also suitable.

In one embodiment, liquid manual dishwashing detergents comprise at least one amphoteric surfactant according to formula (AMS-IIc), wherein preferably R¹¹ is C₁₁, r=3, R¹² and R¹³ are C₁ alkyl, and A⁻ is carboxyl. Preferably, such an amphoteric surfactant is comprised in liquid manual dishwashing detergents in amounts ranging from 1% to 10% by weight, 2% to 8% by weight, or 3% to 6% by weight, all relative to the total weight of the liquid manual dishwashing detergent.

In one embodiment, liquid manual dishwashing detergents comprise at least one amphoteric surfactant according to formula (AMS-IV), wherein preferably R¹⁸ is C₁ alkyl, x is 0 and R¹⁶ is C₁₂. Preferably, such an amphoteric surfactant is comprised in liquid manual dishwashing detergents in amounts ranging from 0.5% to 5% by weight, 1% to 4% by weight, or 1.5% to 3% by weight, all relative to the total weight of the liquid manual dishwashing detergent.

In one embodiment, liquid manual dishwashing detergents comprise at least two anionic surfactants, selected from compounds of general formula (AS-I), wherein one of said anionic surfactants is characterized in R¹ being C₁₁, R² being H, m being 2, n and o=0, A⁻ being SO₃, M⁺ being Na⁺ and the other surfactant is characterized in R¹ being C₁₃, R² being H, m being 2, n and o=0, A⁻ being SO₃⁻, M⁺ being Na⁺. Preferably, such a mixture of anionic surfactants is comprised in liquid manual dishwashing detergents in amounts ranging from 4% to 12% by weight, 5% to 11% by weight, or 6% to 10% by weight, all relative to the total weight of the liquid manual dishwashing detergent.

In one embodiment, liquid manual dishwashing detergents of the invention comprise at least one hydrolase (component (c), preferably comprising a catalytic triad having the motif serine-histidine-aspartate as disclosed herein) as disclosed herein. Preferably, at least one protease as disclosed herein is combined with one or more detergent components by addition of the liquid composition of the invention. Preferably, at least one protease is comprised in amounts of about 0.002% to 0.25% by weight, more preferably about 0.005% to 0.2% by weight, all relative to the total weight of the detergent formulation.

In one embodiment, liquid manual dishwashing detergents additionally comprise at least one alpha-amylase as disclosed herein, preferably in amounts of about 0.001% to 0.005%, more preferably 0.001 to 0.004% by weight, all relative to the total weight of the detergent formulation.

In one embodiment, liquid manual dishwashing detergents comprise at least one ethoxylated polyethylenimine polymer which is based on a polyethylene core and a polyethylene oxide shell. Preferably, polyethylene imine core molecules are polyethylene imines with average molecular weight M_{w} in the range of 500 to 5000 g/mol. More preferred is a molecular weight from 500 to 1000 g/mol, even more preferred is an M_{w} of 600-800 g/mol. The ethoxylated polymer has in average 5 to 50, preferably 10 to 30 and even more preferably 15 to 25 EO (ethoxylate) groups per —NH group, resulting in an average molecular weight M_{w} in the range from 5,000 to 20,0000, preferably 8,000 to 100,000, more preferably 8,000 to 50,000, even more preferably 10,000 to 30,000, and most preferably 10,000 to 20,000) g/mol. Preferably at least one ethoxylated polyethyleneimine polymer is comprised in amounts ranging from about 0.1% to 3% by weight, from about 0.5% to 2.5%, or from about 1% to 2% by weight, all relative to the total weight of the liquid manual dishwashing detergent.

In one embodiment, detergent formulations comprising the components of the liquid compositions of the invention are liquid automated dishwashing detergents. The liquid compositions of the invention are preferably comprised in liquid automated dishwashing detergents in a weight ratio liquid composition:detergent of about 1:1000, 1:500, 1:100, 1:50, 1:30, 1.25, 1:20, or 1:10.

Usually, automated dishwashing detergents do not comprise anionic surfactants. Preferably, liquid automated dishwashing detergents of the invention comprise at least one non-ionic surfactant according to formula (NIS-II), more preferably wherein R¹ is n-C₈ alkyl, R² is branched C₁₁ alkyl, AO is CH₂—CH₂—O, and x is 22. The automated dishwashing detergents preferably comprise such compounds in amounts in the range of about 0.3% to 10% by weight, in the range of about 0.5% to 5% by weight, or in the range of about 1% to 3%, all relative to the total weight of the liquid automated dishwashing detergent.

In one embodiment, liquid automated dishwashing detergents comprise at least one aminocarboxylate as disclosed above in amounts of 5% to 15% by weight relative to the total weight of the detergent formulation.

Preferably, automated dishwashing detergents comprises a builder system comprising
ethylenediaminetetraacetic acid (EDTA) and/or diethylenetriaminepentaacetic acid (DTPA) and/or methylglycine diacetate (MGDA) and/or glutamic acid diacetate (GLDA), as disclosed above in amounts in the range of 0.1% to 15% by weight, in the range of 1% to 10% by weight, in the range of 3% to 8% by weight, or in the range of 2.5% to 5% by weight all relative to the total weight of the detergent formulation;
optionally citric acid in amounts in the range of 0.1% to 10% by weight, in the range of 0.5% to 8% by weight, in the range of 1% to 5% by weight, or in the range of 2% to 4% by weight, all relative to the total weight of the detergent formulation; the citric acid in one embodiment is provided as a mixture with formiate, e.g. Na-citrate:Na-formiate=9:1;
optionally at least one phosphonate, preferably selected from derivatives polyphosphonic acids such as of diphosphonic acid such as sodium salt of HEDP, and derivatives of aminopolyphosphonic acid such as aminoalkylene phosphonic acids such as DTPMP in amounts in the range of 0.1% to 5% by weight, in the range of 0.5% to 3% by weight, or in the range of 1% to 2% by weight, all relative to the total weight of the detergent formulation;
optionally at least one polycarboxylate selected from homopolymers with the repeating monomer being the same unsaturated carboxylic acid, such as polyacrylic acid (PAA) and copolymers with the repeating monomers being at least two different unsaturated carboxylic acids, such as copolymers of acrylic acid with methacrylic acid, copolymers of acrylic acid or methacrylic acid and maleic acid and/or fumaric acid, in amounts in the range of 0% to 10% by weight, 0.5% to 7% by weight, 1% to 5% by weight, or 2.5% to 5% by weight, all relative to the total weight of the detergent formulation; said homopolymers of poly acrylic acid may be partially neutralized or sulfonated.

In one embodiment, liquid automated dishwashing detergents of the invention, comprise at least one hydrolase (component (c), preferably comprising a catalytic triad having the motif serine-histidine-aspartate as disclosed herein) as disclosed herein. At least one protease as disclosed herein is combined with one or more detergent components by addition of the liquid composition of the invention. Preferably, at least one protease is comprised in amounts of about 0.10% to 0.25% by weight, more preferably about 0.12% to 0.21% by weight, all relative to the total weight of the detergent formulation.

In one embodiment, liquid automated dishwashing detergents additionally comprise at least one alpha-amylase as disclosed herein, preferably in amounts of about 0.002% to 0.015%, more preferably 0.004 to 0.01% by weight, all relative to the total weight of the detergent formulation.

In one embodiment liquid automatic dishwashing detergents comprise at least one zinc salt. Zinc salts are preferably selected from water-soluble and water-insoluble zinc salts. In this connection, within the context of the present invention, water-insoluble is used to refer to those zinc salts which, in distilled water at 25° C., have a solubility of 0.1 g/I or less. Zinc salts which have a higher solubility in water are accordingly referred to within the context of the present invention as water-soluble zinc salts.

The zinc salt may be selected from zinc benzoate, zinc gluconate, zinc lactate, zinc formate, ZnCl2, ZnSO4, zinc acetate, zinc citrate, Zn(NO3)₂, Zn(CH3SO3)₂ and zinc gallate, preferably ZnCl2, ZnSO4, zinc acetate, zinc citrate, Zn(NO3)2, Zn(CH3SO3)2 and zinc gallate.

In another embodiment of the present invention, zinc salt is selected from ZnO, ZnO·aq, Zn(OH)2 and ZnCO3. Preference is given to ZnO·aq.

In one embodiment of the present invention, zinc salt is selected from zinc oxides with an average particle diameter (weight-average) in the range from 10 nm to 100 μm.

The cation in zinc salt can be present in complexed form, for example complexed with ammonia ligands or water ligands, and in particular be present in hydrated form. To simplify the notation, within the context of the present invention, ligands are generally omitted if they are water ligands.

Depending on how the pH of mixture according to the invention is adjusted, zinc salt can change. Thus, it is for example possible to use zinc acetate or ZnCl2 for preparing formulation according to the invention, but this converts at a pH of 8 or 9 in an aqueous environment to ZnO, Zn(OH)2 or ZnO·aq, which can be present in non-complexed or in complexed form.

Zinc salt preferably is present in liquid detergent formulations in dissolved or in colloidal form.

In one embodiment of the present invention, inventive automatic dishwashing formulations comprise in total in the range from 0.05 to 0.4% by weight of zinc salt, based in each case on the solids content of the formulation in question. Herein, the fraction of zinc salt is given as zinc or zinc ions. From this, it is possible to calculate the counterion fraction.

Enzyme Stabilization in Detergent Formulation

In one aspect of the invention, component (c) is storage-stable within a liquid detergent formulation of the invention (which may be called in-detergent storage stability herein). Preferably liquid detergent formulations of the invention comprise at least one surfactant as disclosed above. In a preferred embodiment, the detergent formulation is a formulation selected from laundry detergent, automated dishwashing detergent and manual dishwashing detergent. The specific features of said detergent formulations are as disclosed above.

Storage-stability relating to hydrolase activity, preferably proteolytic activity and/or lipolytic activity, within a detergent formulation (which may be called in-detergent storage stability herein), preferably means that the residual hydrolytic activity after storage for up to 28 days, up to 42 days, or up to 84 days at elevated temperatures of about 30° C. or 37° C. is at least 60%, at least 65%, at least 70%, at least 75%, at least 80%, or at least 85% when compared to the initial hydrolytic activity available before storage. Preferably, component (c) comprises at least one hydrolase comprising as a catalytic triad the amino acids aspartate, histidine and serine. Preferably, at least one hydrolase is a subtilisin proteases (EC 3.4.21.62), preferably a protease according to SEQ ID NO:22 as described in EP 1921147 or variants thereof having proteolytic activity, preferably a protease 80% identical to SEQ ID NO:22 as described in EP 1921147 having R101E.

In one embodiment, the in-detergent storage stability of at least one protease (preferably at least one subtilisin type proteases (EC 3.4.21.62)) refers to residual proteolytic activity in a detergent formulation of the invention comprising the liquid composition of the invention and 5% to 15% by weight relative to the total weight of the detergent formulation of at least one aminocarboxylate (preferably MGDA). Preferably said detergent formulation further comprises up to 5% by weight relative to the total weight of the detergent formulation of at least one NIS-II (preferably about 2% by weight of at least one NIS-II) and/or 0.1% to 5.0% by weight relative to the total weight of the detergent formulation of at least one phosphonate (preferably HEDP). The residual proteolytic activity after 28 days of storage at 37° C. in said detergent formulation is preferably at least 40% or at least 50% or at least 60% or at least 65% of at least 70% or at least 75% when compared to initial proteolytic activity available before storage

In one embodiment, the in-detergent storage stability of at least one protease (preferably at least one subtilisin type proteases (EC 3.4.21.62)) refers to residual proteolytic activity in a detergent formulation of the invention comprising the liquid composition of the invention and at least one anionic surfactant such as AS-I and/or AS-II and/or soap, preferably together with at least one non-ionic surfactant. Preferably anionic surfactants are comprised in amounts ranging from 4% to 12% by weight relative to the total weight of the detergent formulation. Preferably said detergent formulation further comprises 2% to 5% by weight relative to the total weight of the detergent formulation of at least one aminocarboxylate (preferably MGDA). The residual proteolytic activity after 28 days of storage at 30° C. in said detergent formulation is preferably at least 40% or at least 50% or at least 60% or at least 65% of at least 70% or at least 75% or at least 80% or at least 85% when compared to initial proteolytic activity available before storage.

In one aspect of the invention, the in-detergent storage stability of at least one hydrolase, preferably in-detergent storage stability of protease and/or lipase, comprised in a liquid detergent formulation of the invention, is increased in the presence of at least one compound according to formula (A), preferably triethyl citrate, when compared to the in detergent-stability in liquid detergents lacking said compound. Preferably, at least one compound according to formula (A), preferably triethyl citrate is present in amounts of about 0.2% to 1% by weight, 0.3% to 0.9% by weight, 0.4% to 0.8% by weight, or 0.5% to 0.7% by weight, all relative to the total weight of the detergent formulation.

In one embodiment, the residual hydrolytic activity, preferably proteolytic activity and/or lipolytic, in liquid detergent formulations of the invention comprising triethyl citrate after storage at elevated temperatures of about 30° C. or 37° C. for up to 42 days is at least 95% when compared to the initial enzymatic activity available before storage. Increase of storage stability in the presence of triethyl citrate preferably means increase in residual enzymatic activity after storage by at least 30%, at least 25%, at least 20%, at least 15%, or at least 10% when compared to liquid detergent formulations lacking triethyl citrate.

In one embodiment, the residual hydrolytic activity, preferably proteolytic activity and/or lipolytic activity, in liquid detergent formulations of the invention comprising triethyl citrate after storage at elevated temperatures of about 30° C. or 37° C. for up to 84 days is at least 85% when compared to the initial enzymatic activity available before storage.

Wash Performance in Detergent Formulation

The invention relates to the use of liquid detergent formulations of the invention to remove stains and/or soils, preferably proteinaceous stains from surfaces to be cleaned such as textiles and hard surfaces.

Wash performance relates to the ability to remove stains and/or soils under relevant cleaning conditions.

The term “laundering” relates to both household laundering and industrial laundering and means the process of treating textiles with a solution comprising a detergent formulation of the present invention. The laundering process usually is carried out by using technical devices such as a household or an industrial washing machine. Alternatively, the laundering process can be done by hand.

The term “textile” means any textile material including yarns (thread made of natural or synthetic fibers used for knitting or weaving), yarn intermediates, fibers, non-woven materials, natural materials, synthetic materials, as well as fabrics (a textile made by weaving, knitting or felting fibers) made of these materials such as garments (any article of clothing made of textile), cloths and other articles.

The term “fibers” includes natural fibers, synthetic fibers, and mixtures thereof. Examples of natural fibers are of plant (such as flax, jute and cotton) or animal origin, comprising proteins like collagen, keratin and fibroin (e.g. silk, sheeps wool, angora, mohair, cashmere). Examples for fibers of synthetic origin are polyurethane fibers such as Spandex® or Lycra®, polyester fibers, polyolefins such as elastofin, or polyamide fibers such as nylon. Fibers preferably include single fibers or parts of textiles such as knitwear, wovens, or nonwovens.

The term “hard surface cleaning” is defined herein as cleaning of hard surfaces wherein hard surfaces include any hard surfaces in the household, such as floors, furnishing, walls, sanitary ceramics, glass, metallic surfaces including cutlery or dishes. The term “hard surface cleaning” in one aspect means “dish washing” which refers to all forms of washing dishes, e.g. by hand or automatic dish wash (ADW). Dish washing includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics.

The inventive washing and/or cleaning process is being carried out at temperatures in the range of from 10° C. to 90° C. In embodiments wherein the inventive cleaning process is carried out as a laundering process, it is preferably carried out at a temperature in the range of from 10° C. to 60° C., more preferably 20° C. to 40° C. In embodiments wherein the inventive cleaning process is carried out as an automatic dishwashing process, it is preferably carried out at a temperature in the range of from 45° C. to 65° C., more preferably 50° C. to 60° C. Said temperatures refer to the temperature of the washing and/or cleaning water being used in the inventive process.

In one embodiment, the use of the detergent formulations of the invention increases the wash performance towards proteinaceous stains and/or soils comprising material such as blood, egg and/or milk, when compared to detergent formulations lacking at least one protease as disclosed in chapter relating to component (c) herein. Preferably, when compared to detergent formulations lacking the components of the liquid composition of the invention.

Preferably, the use of automated dishwashing detergents according to the invention increases the wash performance towards crème brûlée, more preferably when attached to porcelain surfaces, when compared to detergent formulations lacking at least one protease as disclosed in chapter relating to component (c) herein. Preferably, when compared to detergent formulations lacking the components of the liquid composition of the invention.

### Examples

The invention will be further illustrated by the following working examples.

General remarks: percentages are weight percent unless specifically noted otherwise.

I. Storage Stability of Liquid Compositions at 4° C.—Product Homogeneity

Liquid compositions (LC) have been produced by mixing of the following ingredients:

| LC- . . . | LC-I | LC-II | LC-III | LC-IV |
|---|---|---|---|---|
| | wt % in formulation | | | |
| Peptide aldehyde | 0.3 | 0.3 | 0.3 | 0.25 |
| MPG | 35 | 53 | 60 | 57 |
| Hydrolase | 4.5 | 4.5 | 4.5 | 5 |
| CaCl₂*2H₂O | 0.3 | 0.3 | 0.3 | 0.3 |

| | |
|---|---|
| balance | Water up to 100% |

Peptide aldehyde used in the examples is a compound according to formula (PA), wherein Z=Cbz, R¹ is a group such that NH—CHR¹—CO is Val, R² is a group such that NH—CHR²—CO is Ala, and R³ is a group such that NH—CHR³—CO is Leu). The amount of the tripeptide aldehyde relates to 100% active content.

MPG=1,2-propane diol; the amounts displayed in the tables correspond to 100% active content.

Hydrolase used in the examples (LC- . . . ): SEQ ID NO:22 as described in EP 1921147 having the mutation R101E (according to BPN′ numbering). Hydrolase was added in amounts of . . . % by weight as indicated in the tables above and below, wherein the content relates to 100% enzyme activity.

LC-I, LC-II, LC-III and LC-IV were stored at 4° C. Visual evaluation resulted in observation of turbidity and visible particles within 72 h.

| | LC-. . . | | | | | | |
|---|---|---|---|---|---|---|---|
| | LC-V | LC-VI | LC-VII | LC-VIII | LC-IX | LC-X | LC-XI |
| | wt % in formulation | | | | | | |
| Peptide aldehyde | 0.3 | 0.3 | 0.3 | 0.3 | 0.3 | 0.4 | 0.4 |
| MPG | 40 | 35 | 30 | 20 | 10 | 30 | 20 |
| 1,2-pentane diol° | 10 | 15 | 20 | 30 | 40 | 20 | 30 |
| Hydrolase | 4.5 | 4.5 | 4.5 | 4.5 | 4.5 | 6 | 6 |
| Optical evaluation | − | ++ | ++ | ++ | ++ | + | + |
| Total organic | 50 | 50 | 50 | 50 | 50 | 50 | 50 |
| solvent content | | | | | | | |

CaCl₂2H₂O was added in amounts of 0.3% by weight. Amount by weight relates to the total weight of the liquid composition. Water was added up to 100%.
“diol (other than 1,2-propane diol)”; the amounts displayed in the tables correspond to 100% active content.

Optical evaluation result after storage at 4° C.—scheme as used in the examples:
−: turbidity and/or phase separation
+: remains clear, no phase separation for 10-50 days
++: remains clear, no phase separation for >50 days (up to 199 days)

LC-. . .

| | | | |
|---|---|---|---|
| LC-XII | LC-XIII | LC-XIV | LC-XV |

wt % in formulation

| | | | | |
|---|---|---|---|---|
| Peptide aldehyde | 0.3 | 0.3 | 0.3 | 0.3 |
| MPG | 25 | 20 | 15 | 10 |
| 1,2-pentane diol° | 20 | 20 | 20 | 20 |
| Hydrolase | 4.5 | 4.5 | 4.5 | 4.5 |
| Optical evaluation | ++ | ++ | − | − |
| Total organic | 45 | 40 | 35 | 30 |
| solvent content | | | | |

| | LC-. . . | | |
|---|---|---|---|
| | LC-VI | LC-XVII | LC-XVIII |
| | wt % in formulation | | |
| Peptide aldehyde | 0.3 | 0.3 | 0.3 |
| MPG | 40 | 30 | 20 |
| 1,2-hexane diol° | 10 | 20 | 30 |
| Hydrolase | 5 | 5 | 5 |
| Optical evaluation | ++ | ++ | ++ |
| Total organic | 50 | 50 | 50 |
| solvent content | | | |

| | LC-. . . | | | | | | | |
|---|---|---|---|---|---|---|---|---|
| | LC-XIX | LC-XX | LC-XXI | LC-XXII | LC-XXIII | LC-XXIV | LC-XXV | LC-XXVI |
| | wt % in formulation | | | | | | | |
| Peptide aldehyde | 0.3 | 0.3 | 0.3 | 0.3 | 0.28 | 0.24 | 0.2 | 0.32 |
| MPG | 30 | 20 | 40 | 20 | 50 | 52 | 49 | 50 |
| 1,6-hexane diol° | 30 | 40 | 10 | 30 | 7.5 | 4.5 | 5 | 10 |
| Hydrolase | 4.5 | 4.5 | 4.5 | 4.5 | 5 | 4.5 | 6 | 5 |
| Optical evaluation | − | − | − | − | ++ | ++ | ++ | ++ |
| Total organic | 60 | 60 | 50 | 50 | 57.5 | 56.5 | 54 | 60 |
| solvent content | | | | | | | | |

| | LC-. . . | | | | | | | |
|---|---|---|---|---|---|---|---|---|
| | LC-XXVII | LC-XXVIII | LC-XXIX | LC-XXX | LC-XXXI | LC-XXXII | LC-XXIII | LC-XXXIV |
| | wt % in formulation | | | | | | | |
| Peptide aldehyde | 0.32 | 0.275 | 0.275 | 0.275 | 0.25 | 0.2 | 0.175 | 0.1 |
| MPG | 52.5 | 45 | 50 | 50 | 52.5 | 50 | 48.5 | 50 |
| 1,6-hexane diol° | 7.5 | 10 | 7.5 | 7.5 | 4.5 | 5 | 5 | 2 |
| Hydrolase | 5 | 5 | 4.5 | 5 | 5 | 6 | 6 | 6 |
| Optical evaluation | ++ | ++ | ++ | ++ | ++ | ++ | ++ | ++ |
| Total organic | 60 | 55 | 57.5 | 57.5 | 57 | 55 | 53.5 | 52 |
| solvent content | | | | | | | | |

LC-. . .

| wt % in formulation | LC-XXXV | LC-XXXVI |
|---|---|---|
| Peptide aldehyde | 0.28 | 0.28 |
| MPG | 20 | 50 |
| diethylene glycol° | 30 | 5 |
| Hydrolase | 5 | 5 |
| Optical evaluation | − | ++ |
| Total organic | 50 | 55 |
| solvent content | | |

II. Enzyme Stability in Detergent Formulation

The liquid compositions of I. have been added to detergent formulations (OF) according to Table 3.

TABLE 3
liquid detergent formulation

| Detergent formulation: | DF-I. | | DF-II. | |
|---|---|---|---|---|
| Ingredients | wt % in formulation | | wt % in formulation | |
| MGDA | | 15 | | — |
| Sodium citrate | | 5 | DF-II.a | 3 |
| dihydrate | | | DF-II.b . . . | — |
| PAA | | 5 | | — |
| solvent | G: | 5 | MPG: | 6 |
| | | | C₂H₅OH: | 2 |
| surfactant | S1: | 2 | S2: | 5.5 |
| | | | S3: | 2.4 |
| | | | S4: | 5.4 |
| | | | S5: | 5.4 |
| Na₄HEDP | | 1 | | |
| Xanthan gum | | 0.3 | | — |
| Liquid composition | | 3.5 | | 2.0 |
| LC- . . . * | | | | |

| | | |
|---|---|---|
| Further components | as indicated in examples | as indicated in examples |
| balance | Water up to 100 after adjusting | Water up to 100 after adjusting |
| | pH 9 with citric acid | pH 8-8.7 with NaOH |

* . . . as disclosed in the tables in examples section I PAA, Polyacrylic acid Mw 5.000 g/mol (homo-polyacrylic acid) Solvent: G = glycerol; MPG = 1,2-propane diol

Surfactant:

S1: non-ionic surfactant according to formula (NIS-II), wherein R¹ is n-C₈ alkyl, R² is branched C₁₁ alkyl, AO is CH₂—CH₂—O, and x is 22.

S2: linear alkylbenzene sulfonate according to formula (AS-II), wherein one of said anionic surfactants is characterized in R¹ being C₁₀, and the other surfactant is characterized in R¹ being C₁₃.

S3: Edenor coco fatty acid (C₁₂-C₁₈).

S4: mixture of two non-ionic surfactants according to formula (NIS-I), wherein one of said nonionic surfactants is characterized in R¹ being n-C₁₂H₂₅, R² and R⁵ being H, m being 3-30, preferably 7, n and o=0, and the other surfactant is characterized in R¹ being n-C₁₄H₂₉, R² and R⁵ being H, m being 3-30, preferably 7, n and o=0.

S5: mixture of two anionic surfactant according to formula (AS-I), wherein one of said anionic surfactants is characterized in R¹ being C₁₁, R² being H, m being 2, n and o=0, A⁻ being SO₃, M⁺ being Na⁺ and the other surfactant is characterized in R¹ being C₁₃, R² being H, m being 2, n and o=0, A⁻ being SO₃⁻, M⁺ being Na⁺.

The liquid detergent formulations were stored at a temperature of 30° C. or 37° C. for ≥28 days.

The protease activity (which may be called proteolytic activity herein) before and after storage was analyzed by measuring the reactivity towards the peptidic substrate Suc-AAPF-pNA. Here pNA is cleaved from the substrate molecule at 30° C., pH 8.6 using 100 mM TRIS buffer. The rate of cleavage, directly proportional to the protease activity, can be determined by the increase of the yellow color of free pNA in the solution by measuring OD₄₀₅, the optical density at 405 nm.

The liquid composition LC-XXV was tested for storage stability of protease in the detergent formulation DF-I. for 28 days at 37° C. The residual proteolytic activity measured after 28 days of storage was 75% when compared to the initial enzymatic activity before storage.

The liquid composition LC-XXV was tested for storage stability of protease in the detergent formulation DF-I. comprising 0.9% triethyl citrate (further component) for 42 days and 84 days at 37° C. The residual proteolytic activity was 99% after 42 days of storage when compared to the initial enzymatic activity before storage. The residual enzymatic activity was 88% after 84 days of storage when compared to the initial enzymatic activity before storage.

The liquid compositions LC-XXIV, LC-XXVIII and LC-XXIX were tested for storage stability of protease in the detergent formulation DF-II.b comprising 0.2% by weight lipase product Lipex® 100L, 0.1% by weight mannanase product Mannaway® 200L, 0.2% by weight cellulase product Celluclean® 5000L and 0.2% by weight amylase product Amplify® Prime 100L (further components; all enzymes from Novozymes) for 28 days at 30° C. In all three cases, the residual proteolytic activity was about 85% after 28 days of storage when compared to the initial enzymatic activity before storage.

III. Wash Performance in Detergent Formulation

The test is performed according to the recommendations for the quality assessment of the cleaning performance of dishwasher detergents from the German Cosmetic, Toiletry, Parfumery and Detergent Association (IKW).

The performance testing is done at 21±1° dH (3.7±0.2 mmol (Ca+Mg) per litre) water hardness in household relevant Miele GSL2 dishwashing machines by using the “R50° C./3 min/KI55° C.” program (normal program) in presence of ballast soil. The maximum temperature during the main wash cycle is 50° C. and the water softener of the machine is set inoperative.

Wash is done in the presence of 50 g of ballast soil to be applied at the beginning of the main wash cycle:

| | % by weight |
|---|---|
| Vegetable oil | 31.6 |
| whole egg | 15.8 |
| whipped cream, 30% | 9.4 |
| margarine, lard, deep frying fat (partly liquid), | 6.3 |
| milk (3.5%), tomato ketchup, mustard | each |
| potato starch | 2.2 |
| gravy | 1.7 |
| wheat flour | 0.6 |
| quark powder | 0.6 |
| Benzoic acid > 99.9% | 0.3 |

Combine vegetable oil and whole egg and mix thoroughly (approx. 30 minutes). Add ketchup and mustard, still stirring vigorously. Melt the fats, allow to cool to approx. 40° C., then add to the mixture and blend in well. Stir in cream and milk. Add the powdered solid constituents and mix everything to a smooth paste. Freeze in 50 g portions.

50 g of frozen ballast soil are added at the same time with 25 ml liquid detergent.

The following protease-sensitive soil types are tested:

| | | |
|---|---|---|
| minced meat | dessert plates, porcelain | Visual assessment |
| egg yolk | stainless steel sheets | Gravimetrical assessment |
| creme brulee | dessert plates, porcelain | Visual assessment |

Soiled items were prepared and assessed according to recommendations for the quality assessment of the cleaning performance of dishwasher detergents from the German Cosmetic, Toiletry, Parfumery and Detergent Association (IKW).

Visual assessments determine the removed soil by means of a photo catalogue representing a 10 point scale from 0 (very poor) to 10 (very good). Gravimetrical assessments determine the removed soil by weighing.

Performance indices:

| Performance index | Definition | Visual score | Gravimetrical score |
|---|---|---|---|
| 0* | No cleaning | 0 | 0% |
| 100 | Total cleaning | 10 | 100% |

*Soils assessed by visual grading reach a lowest performance index of 10

1. A liquid composition, comprising
component (a): at least one peptide aldehyde, and
component (b): at least two organic solvents, wherein one organic solvent is 1,2-propane diol and at least one further organic solvent is selected from the group consisting of diols other than 1,2-propane diol in a weight ratio of 1,2-propane diol: at least one diol other than 1,2-propane diol of 25:1 to 1:4.

2. The composition according to claim 1, wherein the at least one diol other than 1,2-propane diol is selected from the group consisting of alpha-omega diols, 1,2 butane-diol, 1,3-butane diol, 1,2-pentane diol, and 1,2-hexane diol.

3. The composition according to claim 1, wherein in component (b), 1,2-propane diol and the at least one is diol other than 1,2-propane diol are present in a ratio of 25:1 to 4.5:1.

4. The composition according to claim 3, wherein the at least one diol other than 1,2-propane diol is selected from the group consisting of alpha-omega diols.

5. The composition according to claim 1, wherein amounts of the at least one diol other than 1,2-propane diol and component (a) are in a weight ratio of about 40:1 to about 15:1.

6. The composition according to claim 1, wherein the composition additionally comprises
component (c): at least one hydrolase comprising as a catalytic triad the amino acids aspartate, histidine and serine.

7. The composition according to claim 1, wherein
component (b) is comprised in a total amount of about 45% to 65% by weight relative to a total weight of the liquid composition.

8. The composition according to claim 1, wherein component (b) comprises of 1,2-propane diol in amounts of about 10% to 60% by weight relative to a total weight of the liquid composition.

9. A liquid detergent formulation comprising the liquid composition according to claim 1 and at least one detergent component selected from the group consisting of surfactants and non-phosphate based builders.

10. The detergent formulation according to claim 9, wherein the detergent formulation further comprises at least one compound according to formula (A) 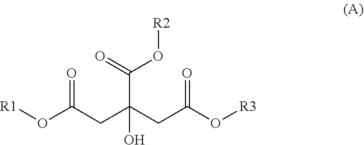
wherein the variables in formula (A) are defined as follows:
R1, R2, R3 are selected from the group consisting of H, linear C₁-C₈ alkyl, and branched C₃-C₈ alkyl, wherein at least one of R¹, R², and R³ is not H.

11. The detergent formulation according to claim 9, further comprising at least one hydrolase, wherein when the detergent is stored for at least 28 days at 30° C., at least one hydrolase has at least 60% residual hydrolytic activity when compared to initial hydrolytic activity of the detergent available before storage.

12. A method of using at least one diol other than 1,2-propane diol selected from the group consisting of alpha-omega diols, 1,2 butane-diol, 1,3-butane diol, 1,2-pentane diol, and 1,2-hexane diol, the method comprising using the at least one diol other than 1,2-propane diol to increase solubility of a tripeptide aldehyde in 1,2-propane diol, wherein the tripeptide aldehyde is selected from the group consisting of compounds according to formula (PA) 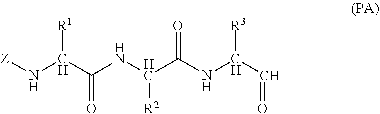
wherein
R¹ and R² are groups such that NH—CHR¹—CO and/or NH—CHR²—CO are non-polar amino acids,
R³ is a group such that NH—CHR³—CO is an L or D-amino acid residue of Tyr, Phe, Val, Ala or Leu;
and
the N-terminal protection group Z is selected from the group consisting of benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), and tert-butyloxycarbonyl (Boc).

13. The method according to claim 12, wherein the resulting liquid product is homogenous at ambient temperatures.

14. A method of using a detergent formulation comprising the liquid composition according to claim 1 and at least one detergent component selected from the group consisting of surfactants and non-phosphate based builders, the method comprising using the detergent formulation to increase wash performance towards proteinaceous stains when compared to use of detergent formulations lacking the components of the liquid composition.

15. A method of using at least one diol other than 1,2-propane diol selected from the group consisting of alpha-omega diols, 1,2 butane-diol, 1,3-butane diol, 1,2-pentane diol, and 1,2-hexane diol, the method comprising using the at least one diol other than 1,2-propane diol to remove turbidity and/or dissolve particles from a liquid composition comprising at least 1,2-propane diol and a tripeptide aldehyde according to formula (PA) 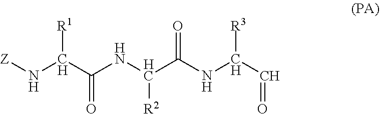
wherein
R¹ and R² are groups such that NH—CHR¹—CO and/or NH—CHR²—CO are non-polar amino acids,
R³ is a group such that NH—CHR³—CO is an L or D-amino acid residue of Tyr, Phe, Val, Ala or Leu;
and
the N-terminal protection group Z is selected from the group consisting of benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), and tert-butyloxycarbonyl (Boc).

16. The liquid composition according to claim 1, wherein the at least one peptide aldehyde is a tripeptide aldehyde selected from the group consisting of compounds according to formula (PA) 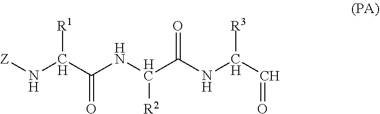
wherein
R¹ and R² are groups such that NH—CHR¹—CO and/or NH—CHR²—CO are non-polar amino acids;
R³ is a group such that NH—CHR³—CO is an L or D-amino acid residue of Tyr, Phe, Val, Ala or Leu;
and
the N-terminal protection group Z is selected from the group consisting of benzyloxycarbonyl (Cbz), p-methoxybenzyl carbonyl (MOZ), benzyl (Bn), benzoyl (Bz), p-methoxybenzyl (PMB), p-methoxyphenyl (PMP), formyl, acetyl (Ac), methyloxy, alkoxycarbonyl, methoxycarbonyl, fluorenylmethyloxycarbonyl (Fmoc), and tert-butyloxycarbonyl (Boc).

17. The composition according to claim 1, wherein in component (b), 1,2-propane diol and the at least one diol other than 1,2-propane diol are present in a ratio of 12:1 to 4.5:1.

18. The composition according to claim 3, wherein the at least one diol other than 1,2-propane diol is 1,6-hexane diol.

19. The method according to claim 12, wherein the resulting liquid product is homogenous at ambient temperatures and retains homogeneity when stored at 4° C., 30° C. or 37° C. for at least up to 50 days.

20. The composition according to claim 1, wherein the composition additionally comprises
component (c): at least one hydrolase comprising as a catalytic triad the amino acids aspartate, histidine and serine, wherein component (a): component (c) are comprised at a molar ratio of about 1:1 to about 20:1.
